(19) United States
(12) Patent Application Publication
(10) Pub. No.: US 2015/0354006 A1
(43) Pub. Date: Dec. 10, 2015

(54) MARKERS FOR ACUTE LYMPHOBLASTIC LEUKEMIA

(71) Applicants: Ho Man CHAN, (US); Nathan P. ENGLUND, San Diego,, CA (US); Levi GARRAWAY, Boston,, MA (US); Min HU, Hi-Tech Park, Pudong New Area Shanghai (CN); Jacob JAFFE, Boston,, MA (US); Gregory KRYUKOV, Cambridge,, MA (US); Jun LIU, San Diego,, CA (US); Xianghui LIU, Hi-Tech Park, Pudong New Area, Shanghai (CN); Rob MCDONALD, Cambridge,, MA (US); Frank Peter STEGMEIER, (US); Zhaofu WANG, Hi-Tech Park, Pudong New Area, Shanghai (CN); Yan WANG, San Diego, CA (US); Haiping WU, (US); Feng YAN, San Diego,, CA (US)

(72) Inventors: Ho Man Chan, Arlington, MA (US); Nathan P. Englund, Valley Center, CA (US); Levi Garraway, Newton, MA (US); Min Hu, Shanghai (CN); Jacob Jaffe, Cambridge, MA (US); Gregory Kryukov, Newton, MA (US); Jun Liu, San Diego, CA (US); Xianghui Liu, Singapore (SG); Rob McDonald, Wayland, MA (US); Frank Peter Stegmeier, Acton, MA (US); Zhaofu Wang, Shanghai (CN); Yan Wang, San Diego, CA (US); Haiping Wu, Shanghai (CN); Feng Yan, LaJolla, CA (US)

(73) Assignees: Novartis AG, Basel (CN); Broad Institute, Inc., Boston, MA (US); Dana-Farber Cancer Institute, Inc., Boston, MA (US)

(21) Appl. No.: 14/434,063

(22) PCT Filed: Oct. 15, 2013
(86) PCT No.: PCT/US13/64976

§ 371 Date: Apr. 7, 2015

(30) Foreign Application Priority Data
Oct. 19, 2012 (CN) PCT/CN2012/083245

(51) Int. Cl. C12Q 1/68; G01N 33/574

(52) U.S. Cl. CPC C12Q 1/6886; G01N 33/57426; C12Q 2600/154; G01N 2333/91017; C12Q 2600/156; G01N 2440/12; C12Q 2600/136; G01N 2500/10; G01N 2500/04

(57) ABSTRACT

The invention provides methods of detecting a NSD2 mutation in a cancer cell, methods cancer diagnosis and methods of screening for NSD2 inhibitors.

### FIELD OF THE INVENTION

The present invention relates to the field of pharmacogenomics, and the use of biomarkers useful in determining patient sensitivity prior to treatment, following patient response after treatment, cancer sensitivity and screening of compounds.

### BACKGROUND

Nuclear SET domain-containing protein (NSD2) is a histone methyltransferase that acts on lysine side chains at amino acid position 36 (K36) of the histone H3 (H3K36). In the normal cell, NSD2 facilitates chromosomal integrity, with the H3K36 methylation shown to be important in gene expression, alternative splicing and DNA repair (Lucio-Eterovic et al., Transcription 2011 2(4):158-161). NSD2 is deleted in patients with Wolf-Hirshhorn syndrome, a disorder characterized by developmental defects, including mental retardation (Stec et al., Hum. Mol. Genet. 1998 7:1071-1082). In patients with the plasma cell disorder of primary systemic amyloidosis, it was discovered that there was a chromosomal translocation of t(4; 14) (p16.3; q32.3), resulting in two fusions—FGFR3 brought under the influence of the Ig gene enhancer Ea and NSD2 under the influence of enhancer Eμ. This specific chromosomal translocation disrupts the regulation of both the NSD2 gene as well as the FGFR-3 gene. Of the 42 primary systemic amyloidois patients assayed for the IGH/NSD2 translocation, 6 patients (14%) contained the mutation. Survival of these 6 patients ranged from a very short period of time (4 months) to fairly long (63 months).

Changing the focus to cancer, the same group found that in multiple myeloma, NSD2 had also undergone a chromosomal translocation at t(4; 14) (p16.3; q32.3). Multiple myeloma patients with the NSD2 translocation have a poorer prognosis than patients who do not have the translocation (Intini et al., Brit. J. Haem. 2004, 126:437-439, see also Malgeri et al., Cancer Res. 2000, 60:4058-4061).

Acute lymphoblastic leukemia (ALL) is a hematopoeitic cancer characterized by malignant, immature lymphblast proliferation in the bone marrow. It is especially prevalent in children, accounting for approximately 30% of all cancers diagnosed in children less than 15 years of age (Linet et al., J. Nat. Cancer Inst. 1999, 91(12):1051-1058). Children with B cell precursor ALL, a low leukocyte count and are 1 to 9 years in age, have a favorable prognosis. In contrast, patient with the t(4;11)/MLL-AF4 fusion are considered to have higher risk ALL, and less chance of survival. (Pui et al., Hematology Am Soc. Hema. Educ. Prog. 2004, 118-45). New markers that provide aid to diagnosing ALL are useful in gaining an early diagnosis and assessment of ALL prognosis.

In this work, the mutational status of NSD2 is used as a biomarker or indicator. Finding biomarkers which indicate which patient should receive a therapeutic is useful, especially with regard to cancer. This allows for more timely and aggressive treatment as opposed to a trial and error approach. In addition, the discovery of biomarkers which indicate that cells continue to be sensitive to the therapy after administration is also useful. These biomarkers can be used to monitor the response of those patients receiving the therapeutic. If biomarkers indicate that the patient has become insensitive to the treatment, then the dosage administered can be increased, decreased, completely discontinued or an additional therapeutic administered. As such, mutations in NSD2 are useful in the diagnosis of ALL. This approach ensures that the correct patients receive the appropriate treatment and during the course of the treatment the patient can be monitored for chemotherapy resistance.

In the diagnosis of ALL, NSD2 biomarkers will aid in understanding the mechanism of action. The mechanism of action may involve a complex cascade of regulatory mechanisms in the cell cycle and differential gene expression. This analysis is done at the pre-clinical stage of drug development in order to determine the particular sensitivity of ALL cancer cells containing a NSD2 mutation and the activity of the therapy.

### SUMMARY OF THE INVENTION

The disclosure is directed to diagnosis of hematological cancer by analysis of histone lysine N-methyltransferase Nuclear SET Domain containing protein 2 (NSD2) mutations. NSD2 is also known as Wolf-Hirschhorn Syndrome Candidate 1 (WHSC1) and Multiple Myeloma SET domain-containing protein II (MMSETII). Specific mutants of NSD2 act as biomarkers in determining the subsets of patients with Acute Lymphoblastic Leukemia (ALL). NSD2 mutational analysis provides a “gene signature” for ALL that has increased accuracy and specificity in segregating ALL cancer patients. The method analyzes a NSD2 mutation in a cancer sample taken from a patient and then compared to a non-mutant or wild-type control. The pattern of NSD2 mutation can be indicative of a favorable response or an unfavorable one. The invention is an example of “personalized medicine” wherein patients are treated based on a functional genomic signature that is specific to that individual.

The predictive value of NSD2 mutations can also be used after treatment with a cancer therapy to determine if the patient is remains sensitive to the treatment. Once a therapeutic has been administered, a NSD2 mutation can be assayed for to monitor the continued sensitivity of the patient to the therapy. This is useful in determining that patients receive the correct course of treatment. The disclosure comprises a method of determining if a patient has a specific type of ALL.

### DESCRIPTION OF THE DRAWINGS

FIG. 1 shows that tumorigenesis in a mouse model of multiple myeloma is dependent on a NSD2 t(4,14) breakpoint alteration.

FIG. 2A shows that a NSD2 knockout inhibited tumorigenesis in a mouse model of multiple myeloma.

FIG. 2B shows the changes in body weight in a mouse model of multiple myeloma containing a t(4,14) breakpoint alteration, wherein NSD2 is knocked out.

FIG. 3A shows that an inducible shRNA knockdown of NSD2 inhibited tumorigenesis in a mouse model of multiple myeloma with a t(4,14) breakpoint.

FIG. 3B shows the difference in luminescence of tumors in a mouse model of multiple myeloma with a NSD2 t(4,14) breakpoint when treated with doxycycline to induce shRNA knockdown of NSD2.

FIG. 4 shows that in a mouse model of multiple myeloma, inducible shRNA knockdown of NSD2 reduces the number of mice with paralysis.

FIG. 5A is a graphic of the NSD2 protein pointing out the E1099K mutation, in alignment with the methyltransferase EZH2.

FIG. 5B is a graphic representation of the NSD2 E1099K mutation.

FIG. 6A shows the sequencing and the characterization of cell lines containing the NSD2 E1099K mutant.

FIG. 6B shows the subtype of ALL and the number of E1099K mutations found.

FIGS. 7A and 7B are crystal structure models showing a NSD2 E1099K mutation in relation to the NSD2 substrate pocket.

FIG. 8 shows the gain of function activity of a NSD2 E1099K mutant.

FIG. 9 shows a LC-MS experiment of a NSD2 E1099K mutant demonstrating increased activity.

FIG. 10 is a Western blot of KMS11 NSD2 knockout cells, reconstituted with NSD2 E1099K mutant.

FIG. 11 depicts a Western blot of H3K36, H3K27, H3K4 and H3K9 methylation status in KMS11 NSD2 knockout cells, rescued with the NSD2 E1099K mutant.

FIG. 12 is the result of assays of KMS11 NSD2 knockout cells reconstituted with the NSD2 E1099K mutant showing that the NSD2 E1099K mutation increases cell proliferation and colony formation.

FIG. 13 shows Western blotting of ALL cell lines wherein NSD2 has been knocked down by inducible shRNA.

FIG. 14 shows the results of a cell proliferation assay of ALL cell lines that are positive or negative for the E1099K mutation, and the effect of NSD2 knockdown by inducible shRNA.

FIG. 15 shows that leukemia cells containing the E1099K mutation have increased H3K36 methylation and reduced levels of H3K27 methylation levels, when compared to KMS11 cells containing the NSD2 t(4,14) breakpoint.

FIG. 16 shows that shRNA knockdown of NSD2 in leukemia cell lines results in the reduction of H3K36 methylation and increase in H3K27 methylation.

FIG. 17 is a table depicting a soft agar colony formation assay of ALL cells containing the E1099K mutation.

FIG. 18 is a heat map of histone profiling at H3K36/H3k27 showing profiles between multiple myeloma cells containing the t(4,14) breakpoint and cells containing the E1099K mutation.

FIG. 19A is a Western Blot of the methylation profile of H3K36, and FIG. 19B is a Western blot of the H3K27 methylation profile.

### DESCRIPTION OF THE INVENTION

The disclosure is directed to methods of diagnosing a patient by analyzing a NSD2 mutation in a sample containing cancer cells wherein the presence of a NSD2 mutation when compared to a non-mutated or wild type control indicates the patient has ALL. The NSD2 mutation of a glutamic acid to lysine change at amino acid position 1099 (E1099K) can be indicative of a favorable patient response or of an unfavorable one and patients can be selected or rejected based on the presence of a NSD2 E1099K mutation.

A method of detecting a cancer cell, the method comprising;

a) obtaining a cancer sample from a patient; b) screening for the presence of a Nuclear SET domain-containing protein (NSD2) mutation; and c) comparing the NSD2 mutation to wild-type NSD2 in a non-cancerous or normal patient sample.

The method wherein the NSD2 mutation is a glutamic acid to lysine change at amino acid position 1099 (E1099K).

The method wherein the cancer cell is a leukemia cell.

The method wherein the leukemia cell is selected from the group consisting of: acute lymphoblastic T cell leukemia, acute lymphoblastic B cell leukemia, plasma cell myeloma, diffuse large B cell lymphoma, and follicular carcinoma

A method of diagnosing cancer, the method comprising; a) obtaining a cancer sample from a patient; b) screening for the presence of a NSD mutation; c) comparing the NSD2 mutation to wild-type NSD2; and d) comparing the methylation of histone H3 at lysine 36 (H3K36) in the cancer sample with a NSD2 mutation, with the methylation of histone H3 at lysine 36 (H3K36) of a non-cancerous or normal patient sample, and increased methylation at H3K36 is indicative of cancer.

The method wherein the NSD2 mutation is E1099K.

The method wherein the cancer sample is leukemia.

The method wherein the leukemia sample is selected from the group consisting of: acute lymphoblastic T cell leukemia, acute lymphoblastic B cell leukemia, plasma cell myeloma, diffuse large B cell lymphoma.

The method, wherein H3K36 is mono-methylated (H3K36me1), di-methylated (H3K36me2) or tri-methylated (H3K36me3).

The method further comprising comparing the methylation of histone H3 at lysine 27 (H3K27) in the cancer sample with an NSD2 mutation, with the methylation of histone H3 at lysine 27 (H3K27) of a non-cancerous or normal patient sample, and decreased methylation at H3K27 is indicative of cancer.

The method wherein H3K27 is unmethylated (H3K27me0), mono-methylated (H3K27me1), di-methylated (H3K27me2) or tri-methylated (H3K27me3).

A method of screening for a NSD2 inhibitor candidate, the method comprising: a) contacting a cell containing a NSD2 mutation with a NSD2 inhibitor candidate; b) measuring the methyltransferase activity; and c) comparing the reduction in methyltransferase activity from the NSD2 mutant cell contacted with the NSD2 inhibitor candidate with methyltransferase activity of the methyltransferase activity of a normal or control cell and/or untreated cells containing the NSD2 mutation.

The method wherein the NSD2 mutation is E1099K.

The method wherein the cell containing a NSD2 mutation is selected from the group consisting of acute lymphoblastic T cell leukemia, acute lymphoblastic B cell leukemia, plasma cell myeloma, diffuse large B cell lymphoma, and follicular carcinoma.

Composition comprising a NSD2 mutation for use in diagnosis of cancer in a selected cancer patient population, wherein the cancer patient population is selected on the basis of containing a NSD2 E1099K mutation in a cancer cell sample obtained from said patients compared to a normal control cell sample.

The composition wherein the cancer sample is selected from the group consisting of is selected from the group consisting of: acute lymphoblastic T cell leukemia, acute lymphoblastic B cell leukemia, plasma cell myeloma, diffuse large B cell lymphoma, and follicular carcinoma.

A kit for predicting the sensitivity of a cancer patient for treatment with a NSD2 inhibitor comprising: i) means for detecting NSD2 E1099K mutation; and ii) instructions how to use said kit.

### DEFINITIONS

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely exemplary and that equivalents of such are known in the art.

The terms “marker” or “biomarker” are used interchangeably herein. A biomarker is a nucleic acid or polypeptide and the presence or absence of a mutation or differential expression is used to determine a specific cancer type. For example, NSD2 is a biomarker in a cancer cell when it is mutated to NSD2 E1099K as compared to NSD2 in normal (non-cancerous) tissue or control tissue.

A cell is “sensitive” or displays “sensitivity” for inhibition with a NSD2 candidate inhibitor when the methyltransferase activity of NSD2 (E1099K) is reduced compared to wild type NSD2 methyltransferase activity.

“NSD2” refers to the histone lysine N-methyltransferase gene. Unless specifically stated otherwise NSD2 as used herein, refers to human NSD2, accession numbers AF071593 (DNA (SEQ ID NO. 1)) and AAC24150.1 (protein (SEQ ID NO.2)).

A “mutant,” or “mutation” is any change in DNA or protein sequence that deviates from wild type NSD2. This includes single base DNA changes, single amino acid changes, multiple base changes in DNA and multiple amino acid changes. This also includes insertions, deletions and truncations of the NSD2 gene and its corresponding protein. For example, a mutation can be a glutamic acid to lysine change at amino acid position 1099 (E1099K) (SEQ ID NO.3).

“RE-IIBP” refers to a truncated isoform of NSD2 generated by alternative splicing and contains a SET domain (Kim et al., Mol Cell Biol. 2008, 28:2023-2034). The sequence has accession number ACE75882.1 (SEQ ID NO. 4)

“MMSET1” refers to a truncated isoform of NSD2 generated by alternative splicing that does not contain a SET domain and has accession number NM_{—}133334 (SEQ ID NO.5)

“Methylation” is the modification of amino acids on a histone protein by the addition of a methyl group. The amino acid can have no methylation (me0), have a single methyl group added (me1), two methyl groups added (me2) or three methyl groups (me3). For example, the nomenclature “H3k36me2” indicates that 2 methyl groups were added to histone H3 at the lysine at position 36. The “methylation status” or “methylation profile” refers to the histone, the amino acid and 0-3 methyl group modifications (me0-me3).

A “control cell,” “normal cell” or “wild-type” refers to non-cancerous cell.

A “control tissue,” “normal tissue” or “wild-type” refers to non-cancerous tissue.

The terms “nucleic acid” and “polynucleotide” are used interchangeably and refer to a polymeric form of nucleotides of any length, either deoxyribonucleotides or ribonucleotides or analogs thereof. Polynucleotides can have any three-dimensional structure and may perform any function. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A “gene” refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

“Gene expression” or alternatively a “gene product” refers to the nucleic acids or amino acids (e.g., peptide or polypeptide) generated when a gene is transcribed and translated.

The term “polypeptide” is used interchangeably with the term “protein” and in its broadest sense refers to a compound of two or more subunit amino acids, amino acid analogs, or peptidomimetics. The subunits can be linked by peptide bonds. In another embodiment, the subunit may be linked by other bonds, e.g., ester, ether, etc.

As used herein the term “amino acid” refers to either natural and/or unnatural or synthetic amino acids, and both the D and L optical isomers, amino acid analogs, and peptidomimetics. A peptide of three or more amino acids is commonly called an oligopeptide if the peptide chain is short. If the peptide chain is long, the peptide is commonly called a polypeptide or a protein.

The term “isolated” means separated from constituents, cellular and otherwise, in which the polynucleotide, peptide, polypeptide, protein, antibody or fragment(s) thereof, are normally associated with in nature. For example, an isolated polynucleotide is separated from the 3′ and 5′ contiguous nucleotides with which it is normally associated within its native or natural environment, e.g., on the chromosome. As is apparent to those of skill in the art, a non-naturally occurring polynucleotide, peptide, polypeptide, protein, antibody, or fragment(s) thereof, does not require “isolation” to distinguish it from its naturally occurring counterpart. In addition, a “concentrated,” “separated” or “diluted” polynucleotide, peptide, polypeptide, protein, antibody or fragment(s) thereof, is distinguishable from its naturally occurring counterpart in that the concentration or number of molecules per volume is greater in a “concentrated” version or less than in a “separated” version than that of its naturally occurring counterpart. A polynucleotide, peptide, polypeptide, protein, antibody, or fragment(s) thereof, which differs from the naturally occurring counterpart in its primary sequence or, for example, by its glycosylation pattern, need not be present in its isolated form since it is distinguishable from its naturally occurring counterpart by its primary sequence or, alternatively, by another characteristic such as glycosylation pattern. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in PCR: A Practical Approach, M. MacPherson et al., IRL Press at Oxford University Press (1991). All processes of producing replicate copies of a polynucleotide, such as PCR or gene cloning, are collectively referred to herein as “replication.” A primer can also be used as a probe in hybridization reactions, such as Southern or Northern blot analyses (Sambrook et al., Molecular Cloning: A Laboratory Manual, 2nd edition (1989)). For example, primers can be designed for use in PCR and subsequent sequencing of exon 20 of human NSD2, Forward Primer: 5′ GTCTGAGATCCTTTGAATTTAATTTATGGA 3′ (SEQ ID NO.6) and Reverse Primer: 5′ GCGCTGCCACAGGGCAAAGTCCAGTTCTAC 3′ (SEQ ID NO.7).

As used herein, “expression” refers to the process by which DNA is transcribed into mRNA and/or the process by which the transcribed mRNA is subsequently translated into peptides, polypeptides or proteins. If the polynucleotide is derived from genomic DNA, expression may include splicing of the mRNA in a eukaryotic cell.

“Differentially expressed” as applied to a gene, refers to the differential production of the mRNA transcribed and/or translated from the gene or the protein product encoded by the gene. A differentially expressed gene may be overexpressed or underexpressed as compared to the expression level of a normal or control cell. However, as used herein, overexpression is an increase in gene expression and generally is at least 1.25 fold or, alternatively, at least 1.5 fold or, alternatively, at least 2 fold, or alternatively, at least 3 fold or alternatively, at least 4 fold expression over that detected in a normal or control counterpart cell or tissue. As used herein, underexpression, is a reduction of gene expression and generally is at least 1.25 fold, or alternatively, at least 1.5 fold, or alternatively, at least 2 fold or alternatively, at least 3 fold or alternatively, at least 4 fold expression under that detected in a normal or control counterpart cell or tissue. The term “differentially expressed” also refers to where expression in a cancer cell or cancerous tissue is detected but expression in a control cell or normal tissue (e.g. non-cancerous cell or tissue) is undetectable.

A high expression level of the gene may occur because of over expression of the gene or an increase in gene copy number. The gene may also be translated into increased protein levels because of deregulation or absence of a negative regulator.

A “gene expression profile” refers to a pattern of expression of at least one biomarker that recurs in multiple samples and reflects a property shared by those samples, such as tissue type, response to a particular treatment, or activation of a particular biological process or pathway in the cells. Furthermore, a gene expression profile differentiates between samples that share that common property and those that do not with better accuracy than would likely be achieved by assigning the samples to the two groups at random. A gene expression profile may be used to predict whether samples of unknown status share that common property or not. Some variation between the levels of at least one biomarker and the typical profile is to be expected, but the overall similarity of the expression levels to the typical profile is such that it is statistically unlikely that the similarity would be observed by chance in samples not sharing the common property that the expression profile reflects.

The term “cDNA” refers to complementary DNA, i.e. mRNA molecules present in a cell or organism made into cDNA with an enzyme such as reverse transcriptase. A “cDNA library” is a collection of all of the mRNA molecules present in a cell or organism, all turned into cDNA molecules with the enzyme reverse transcriptase, then inserted into “vectors” (other DNA molecules that can continue to replicate after addition of foreign DNA). Exemplary vectors for libraries include bacteriophage (also known as “phage”), viruses that infect bacteria, for example, lambda phage. The library can then be probed for the specific cDNA (and thus mRNA) of interest.

As used herein, “solid phase support” or “solid support,” used interchangeably, is not limited to a specific type of support. Rather a large number of supports are available and are known to one of ordinary skill in the art. Solid phase supports include silica gels, resins, derivatized plastic films, glass beads, plastic beads, alumina gels, microarrays, and chips. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories), polyHIPE(R)™ resin (obtained from Aminotech, Canada), polyamide resin (obtained from Peninsula Laboratories), polystyrene resin grafted with polyethylene glycol (TentaGelR™, Rapp Polymere, Tubingen, Germany), or polydimethylacrylamide resin (obtained from Milligen/Biosearch, California).

A polynucleotide also can be attached to a solid support for use in high throughput screening assays. PCT WO 97/10365, for example, discloses the construction of high density oligonucleotide chips. See also, U.S. Pat. Nos. 5,405,783; 5,412,087 and 5,445,934. Using this method, the probes are synthesized on a derivatized glass surface to form chip arrays. Photoprotected nucleoside phosphoramidites are coupled to the glass surface, selectively deprotected by photolysis through a photolithographic mask and reacted with a second protected nucleoside phosphoramidite. The coupling/deprotection process is repeated until the desired probe is complete.

As an example, transcriptional activity can be assessed by measuring levels of messenger RNA using a gene chip such as the Affymetrix® HG-U133-Plus-2 GeneChips (Affmetrix, Santa Clara Calif.). High-throughput, real-time quantitation of RNA of a large number of genes of interest thus becomes possible in a reproducible system.

The terms “stringent hybridization conditions” refers to conditions under which a nucleic acid probe will specifically hybridize to its target subsequence, and to no other sequences. The conditions determining the stringency of hybridization include: temperature, ionic strength, and the concentration of denaturing agents such as formamide. Varying one of these factors may influence another factor and one of skill in the art will appreciate changes in the conditions to maintain the desired level of stringency. An example of a highly stringent hybridization is: 0.015M sodium chloride, 0.0015M sodium citrate at 65-68° C. or 0.015M sodium chloride, 0.0015M sodium citrate, and 50% formamide at 42° C. (see Sambrook, supra). An example of a “moderately stringent” hybridization is the conditions of: 0.015M sodium chloride, 0.0015M sodium citrate at 50-65° C. or 0.015M sodium chloride, 0.0015M sodium citrate, and 20% formamide at 37-50° C. The moderately stringent conditions are used when a moderate amount of nucleic acid mismatch is desired. One of skill in the art will appreciate that washing is part of the hybridization conditions. For example, washing conditions can include 02.×-0.1×SSC/0.1% SDS and temperatures from 42-68° C., wherein increasing temperature increases the stringency of the wash conditions.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region (or a polypeptide or polypeptide region) has a certain percentage (for example, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Current Protocols in Molecular Biology, Ausubel et al., eds., (1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant.

The term “cell proliferative disorders” shall include dysregulation of normal physiological function characterized by abnormal cell growth and/or division or loss of function. Examples of “cell proliferative disorders” includes but is not limited to hyperplasia, neoplasia, metaplasia, and various autoimmune disorders, e.g., those characterized by the dysregulation of T cell apoptosis.

As used herein, the terms “neoplastic cells,” “neoplastic disease,” “neoplasia,” “tumor,” “tumor cells,” “cancer,” and “cancer cells,” (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign. A metastatic cell or tissue means that the cell can invade and destroy neighboring body structures. Cancer can include without limitation: acute lymphoblastic T cell leukemia, acute lymphoblastic B cell leukemia, plasma cell myeloma, diffuse large B cell lymphoma, multiple myeloma and follicular carcinoma.

“Suppressing” tumor growth indicates a reduction in tumor cell growth when contacted with a chemotherapeutic compared to tumor growth without a chemotherapeutic agent. Tumor cell growth can be assessed by any means known in the art, including, but not limited to, measuring tumor size, determining whether tumor cells are proliferating using a 3H-thymidine incorporation assay, measuring glucose uptake by FDG-PET (fluorodeoxyglucose positron emission tomography) imaging, or counting tumor cells. “Suppressing” tumor cell growth means any or all of the following states: slowing, delaying and stopping tumor growth, as well as tumor shrinkage.

A “composition” is a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include pharmaceutical excipients and additives, for example; proteins, peptides, amino acids, lipids, and carbohydrates (e.g., sugars, including monosaccharides and oligosaccharides; derivatized sugars such as alditols, aldonic acids, esterified sugars and the like; and polysaccharides or sugar polymers), which can be present singly or in combination, comprising alone or in combination 1-99.99% by weight or volume. Carbohydrate excipients include, for example; monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like.

The term “carrier” further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-quadrature-cyclodextrin), polyethylene glycols, flavoring agents, antimicrobial agents, sweeteners, antioxidants, antistatic agents, surfactants (e.g., polysorbates such as TWEEN 20™ and TWEEN 80™), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional provisio that they be acceptable for use in vivo. For examples of carriers, stabilizers and adjuvants, see Remington's Pharmaceutical Science., 15th Ed. (Mack Publ. Co., Easton (1975) and in the Physician's Desk Reference, 52nd ed., Medical Economics, Montvale, N.J. (1998).

An “effective amount” is an amount sufficient to effect beneficial or desired results. An effective amount can be administered in one or more administrations, applications or dosages.

A “subject,” “individual” or “patient” is used interchangeably herein, which refers to a vertebrate, preferably a mammal, more preferably a human. Mammals include, but are not limited to, mice, simians, humans, farm animals, sport animals, and pets.

An “inhibitor” of NSD2 as used herein reduces the N-methytransferase activity of NSD2. This inhibition may include, for example, reducing the association of NSD2 and the histone before they are bound together, reducing the association of NSD2 and histone after they are bound together, or binding the NSD2 active site, thus reducing N-methytransferase activity. NSD2 inhibitors are useful in pharmaceutical compositions for human or veterinary use where the NSD2 E1099K mutant is found, e.g., in the treatment of tumors and/or cancerous cell growth. NSD2 inhibitor compounds are useful in treating, for example: acute lymphoblastic T cell leukemia, acute lymphoblastic B cell leukemia, plasma cell myeloma, diffuse large B cell lymphoma, multiple myeloma and follicular carcinoma.

Detection of NSD2 Mutations

The detection of NSD2 mutations can be done by any number of ways, for example: DNA sequencing, PCR based methods, including RT-PCR, microarray analysis, Southern blotting, Northern blotting and dip stick analysis.

The polymerase chain reaction (PCR) can be used to amplify and identify NSD2 mutations from either genomic DNA or RNA extracted from tumor tissue. PCR is well known in the art and is described in detail in Saiki et al., Science 1988, 239:487 and in U.S. Pat. No. 4,683,195 and U.S. Pat. No. 4,683,203.

Methods of detecting NSD2 mutations by hybridization are provided. The method comprises identifying a NSD2 mutation in a sample by contacting nucleic acid from the sample with a nucleic acid probe that is capable of hybridizing to nucleic acid with a NSD2 mutation or fragment thereof and detecting the hybridization. The nucleic acid probe is detectably labeled with a label such as a radioisotope, a fluorescent agent or a chromogenic agent. Radioisotopes can include without limitation; ³H, ³²P, ³³P and ³⁵S etc. Fluorescent agents can include without limitation: fluorescein, texas red, rhodamine, etc.

The probe used in detection that is capable of hybridizing to nucleic acid with a NSD2 mutation can be from about 8 nucleotides to about 100 nucleotides, from about 10 nucleotides to about 75 nucleotides, from about 15 nucleotides to about 50 nucleotides, or about 20 to about 30 nucleotides. The probe or probes can be provided in a kit, which comprise at least one oligonucleotide probe that hybridizes to or hybridizes adjacent to a NSD2 mutation. The kit can also provide instructions for analysis of patient cancer samples that can contain a NSD2 mutation.

Single stranded conformational polymorphism (SSCP) can also be used to detect NSD2 mutations. This technique is well described in Orita et al., PNAS 1989, 86:2766-2770.

Antibodies directed against NSD2 can be useful in the detection of cancer and the detection of mutated forms of NSD2. Antibodies can be generated which recognize and specifically bind only a specific mutant form of NSD2 and do not bind (or weakly bind) to wild type NSD2. These antibodies would be useful in determining which specific mutation was present and also in quantifying the level of NSD2 protein. For example, an antibody can be directed against the glutamic acid to lysine change at amino acid position 1099 (E1099K). An antibody that recognizes this amino acid change and does not specifically bind to wild type NSD2 could identify the specific mutation by Western blotting. Such antibodies can be generated by using peptides containing a NSD2 mutation.

Measurement of Gene Expression

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. For example, using Affymetrix™ U133 microarray chips (Affymax, Santa Clara, Calif.).

In one aspect, gene expression is detected and quantitated by hybridization to a probe that specifically hybridizes to the appropriate probe for that biomarker. The probes also can be attached to a solid support for use in high throughput screening assays using methods known in the art. WO 97/10365 and U.S. Pat. Nos. 5,405,783, 5,412,087 and 5,445,934, for example, disclose the construction of high density oligonucleotide chips which can contain one or more of the sequences disclosed herein. Using the methods disclosed in U.S. Pat. Nos. 5,405,783, 5,412,087 and 5,445,934, the probes of this invention are synthesized on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

In one aspect, the expression level of a gene is determined through exposure of a nucleic acid sample to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device. See U.S. Pat. Nos. 5,578,832 and 5,631,734.

Alternatively any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). However, PCR conditions used for each application reaction are empirically determined. A number of parameters influence the success of a reaction. Among them are annealing temperature and time, extension time, Mg 2+ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination.

In one embodiment, the hybridized nucleic acids are detected by detecting one or more labels attached to the sample nucleic acids. The labels can be incorporated by any of a number of means well known to those of skill in the art. However, in one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g. fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™ Life Technologies, Grand Island, N.Y.), fluorescent dyes (e.g., fluorescein, texas red, rhodamine, green fluorescent protein, and the like), radiolabels (e.g., 3^{H}, 125^{I}, 35^{S}, 14^{C}, or 32^{P}) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the coloured label.

The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

Detection of Polypeptides

A NSD2 mutation when translated into protein can be detected by specific antibodies. A mutation in the NSD2 protein can change the antigenicity, so that an antibody raised against a NSD2 mutant antigen (e.g. a specific peptide containing a mutation) will specifically bind the mutant NSD2 and not recognize the wild-type.

Expression level of a NSD2 mutant can also be determined by examining protein expression. Determining the protein level involves measuring the amount of any immunospecific binding that occurs between an antibody that selectively recognizes and binds to the polypeptide of the biomarker in a sample obtained from a patient and comparing this to the amount of immunospecific binding of at least one biomarker in a control sample. The amount of protein expression of a NSD2 mutant protein can be increased or reduced when compared with control expression. A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), western blot analysis, immunoprecipitation assays, immunofluorescent assays, flow cytometry, immunohistochemistry, confocal microscopy, enzymatic assays, surface plasmon resonance and PAGE-SDS.

Assaying for Biomarkers

Once a patient has been assayed to have a NSD2 mutation, administration of a chemotherapeutic to a patient can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents may be empirically adjusted.

A NSD2 mutation can be assayed for after chemotherapeutic administration in order to determine if the chemotherapeutic treatment remains appropriate. In addition, a NSD2 mutation can be assayed for in multiple timepoints after a single chemotherapeutic administration. For example, after an initial bolus of a chemotherapeutic is administered, a NSD2 mutant can be assayed for at 1 hour, 2 hours, 3 hours, 4 hours, 8 hours, 16 hours, 24 hours, 48 hours, 3 days, 1 week or 1 month or several months after the first treatment.

A NSD2 mutation can be assayed for after each chemotherapeutic administration, so if there are multiple chemotherapeutic administrations, then assaying for a NSD2 mutation after each administration can determine continued course of treatment. The patient could undergo multiple chemotherapeutic administrations and then assayed for a NSD2 mutation at different timepoints. For example, a course of treatment may require administration of an initial dose of chemotherapeutic, a second dose a specified time period later, and still a third dose. A NSD2 mutation could be assayed for at 1 hour, 2 hours, 3 hours, 4 hours, 8 hours, 16 hours, 24 hours, 48 hours, 3 days, 1 week or 1 month or several months after administration of each dose of chemotherapeutic.

Finally, there is administration of different chemotherapeutics, followed by assaying for a NSD2 mutation. In this embodiment, more than one chemotherapeutic is chosen and administered to the patient. A NSD2 mutation can then be assayed for after administration of each different chemotherapeutic. This assay can also be done at multiple timepoints after administration of the different chemotherapeutics. For example, a first chemotherapeutic could be administered to the patient and a NSD2 mutation assayed for at 1 hour, 2 hours, 3 hours, 4 hours, 8 hours, 16 hours, 24 hours, 48 hours, 3 days, 1 week or 1 month or several months after administration. A second chemotherapeutic could then be administered and a NSD2 mutation could be assayed for again at 1 hour, 2 hours, 3 hours, 4 hours, 8 hours, 16 hours, 24 hours, 48 hours, 3 days, 1 week or 1 month or several months after administration of the second chemotherapeutic.

Kits for assessing a NSD2 mutation can be made. For example, a kit comprising nucleic acid primers for PCR or for microarray hybridization for a NSD2 mutation can be used for assessing for ALL. Alternatively, a kit supplied with antibodies for a NSD2 mutation would be useful in assaying for ALL.

Screening for NSD2 Inhibitors

It is possible to use NSD2 mutations to screen for NSD2 inhibitors. This method comprises choosing or engineering a cell with the E1099K NSD2 mutation, the cell is then contacted with the candidate NSD2 inhibitor compound and the contacted cell is assayed. As the NSD2 E1099K mutation shows increased methyltransferase activity, assaying for a reduction in methyltransferase activity when compared to a control cell would indicate that the candidate compound is a NSD2 inhibitor. Alternatively, the contacted cells is assayed for reduction in proliferation or increase in apoptosis. A reduction in proliferation or increase in apoptosis over untreated control sample is a positive result, indicating that the candidate compound inhibits NSD2.

### EXAMPLES

### Example 1

### Tumorigenesis of Multiple Myeloma is NSD2 Dependent

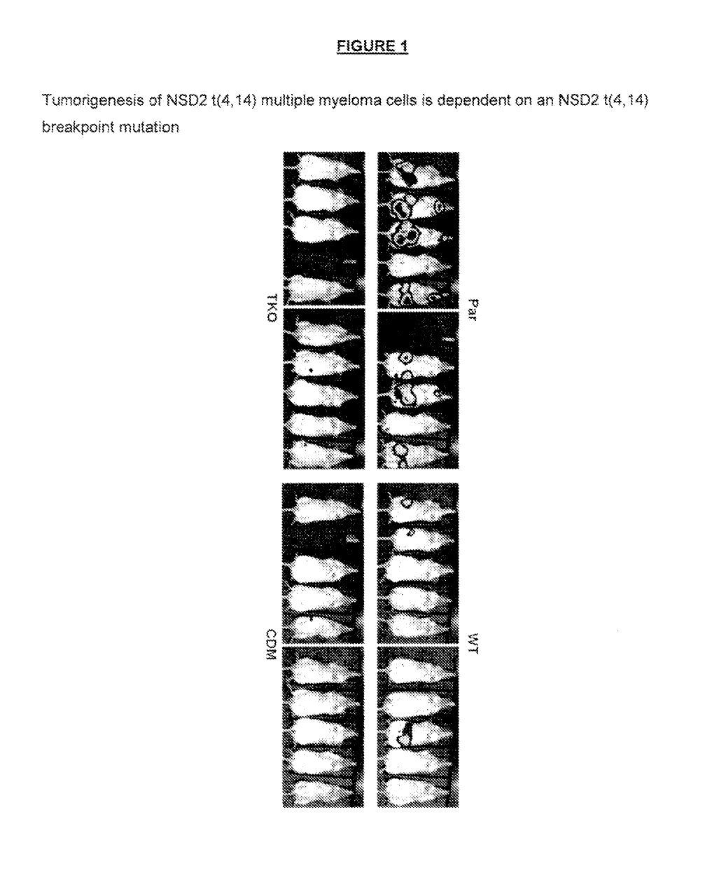

FIG. 1 shows six- to eight-week-old female SCID-beige mice (Vital River, Beijing, China) that were injected intravenously with 10⁷ parental and gene-targeted KMS11 cells. The parental KMS11 cell line is a multiple myeloma line containing t(4:14), and is labeled as “PAR” (parental) in this experiment. A NSD2 knockout (TKO) KMS11 line was obtained from Horizon Discovery Ltd. (Cambridge, UK) and created by deletion of the exon 7 in the t(4,14) NSD2 translocated allele (Lauring et al., Blood 2008 111, 856-864). KMS11-TKO cells were infected with wild type (WT) NSD2, using the 1-1365 isoform of NSD2, and catalytic dead mutant (CDM) NSD2 which has no methyltransferase activity and was created by mutating two amino acids (R1138A/C1144A) (see Marango et al., Blood 2008 111(6):3145-3154 and Kim et al., Mol. Cel. Biol. 2008 6:2023-2034). All KMS11 cells were tagged with luciferase and injected into mice in 100 μl PBS with 50% Matrigel, with 8-10 mice per group/treatment. Images of the mice were taken weekly with D-luciferase (200 μl/mouse) intraperitoneal injection. FIG. 1 shows the tumor growth of KMS11 PAR, TKO, WT and CDM tagged with luciferase by intravenous injection at day 56. The results are that the WT NSD2, but not NSD2 CDM partially rescued the deficiency of NSD2 TKO to form tumors in vivo. This indicates that the tumorigenic potential of KMS11 cells is dependent on NSD2 activity.

### Example 2

### NSD2 Knockout Inhibits Tumor Growth

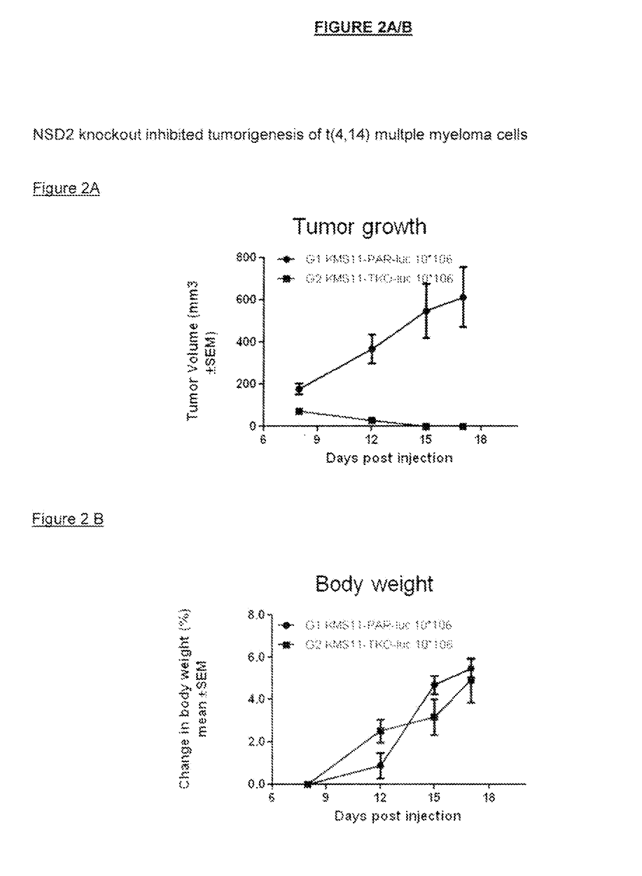

FIG. 2 shows a graph of tumor growth and body weight of six- to eight-week-old female SCID-beige mice (Vital River, Beijing, China) that were injected subcutaneously with 10⁷ parental and gene-targeted KMS11 cells, in 100 μl PBS with 50% Matrigel, 8-10 mice per group/treatment. Tumor volume (½L×W²) and body weight were measured at indicated time on the respective graphs. The result was plotted in FIG. 2(A), which shows the tumor growth curve of KMS11 Par or NSD2 knockout by subcutaneous injection. The KMS11 cells with NSD2 knocked out completely lost tumorigenic potential. FIG. 2(B) shows body weight of mice subcutaneously injected with KMS11 Par or TKO. Note that the KMS11 TKO cells did not form tumors. This reinforces the hypothesis that NSD2 is important in tumorigenesis.

### Example 3

### shRNA Knockdown of NSD2 Inhibits Tumorigenesis

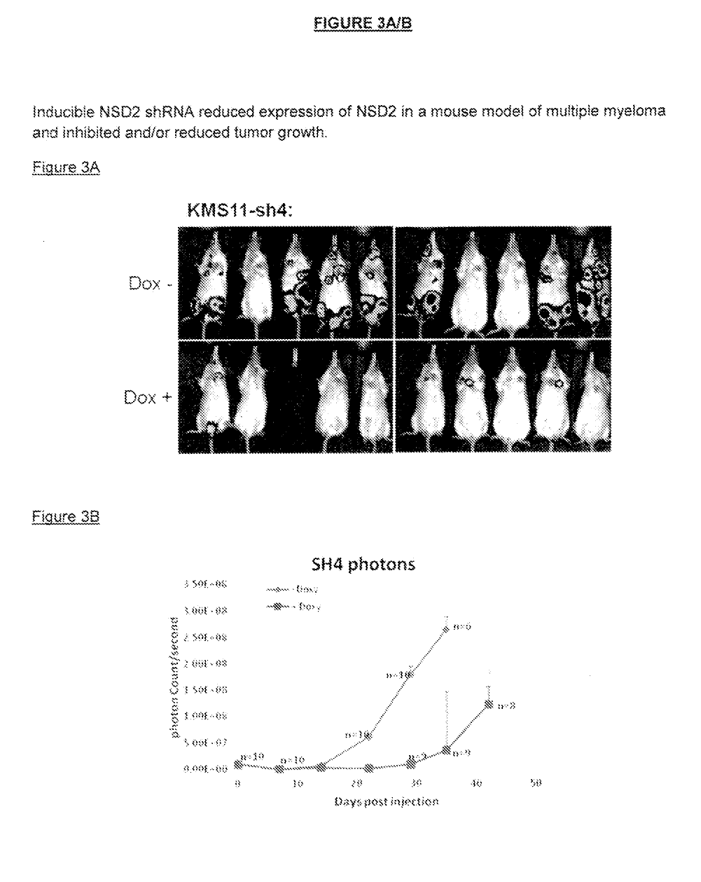

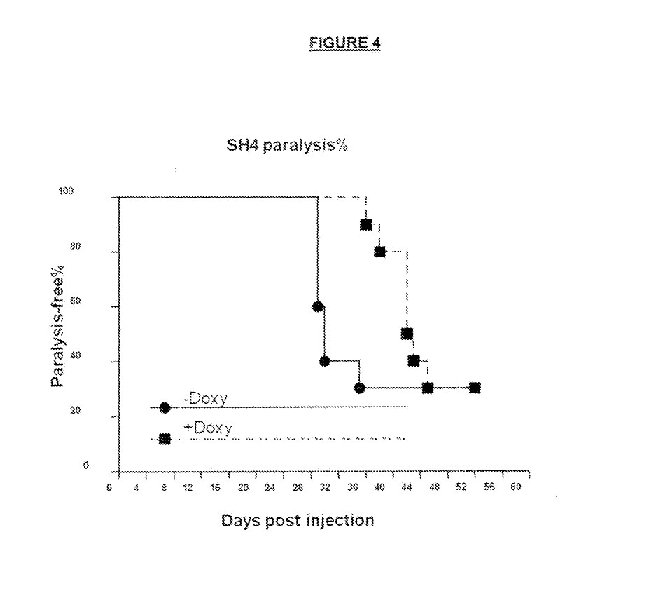

Six- to eight-week-old female SCID-beige mice (Vital River, Beijing, China) were injected intravenously with 10⁷ KMS-11 cells stably transduced with inducible NSD2 shRNA (sh4: 5′ ACATGCTCACTATAGACAA′3 (SEQ ID NO:8)) and tagged with luciferase. The injection of KMS11 cells was in 100 μl PBS with 50% Matrigel, with 8-10 mice per group/treatment. To induce the shRNA expression, mice were fed with water containing 5% sucrose and 0.2 mg/ml doxycycline. Images were taken weekly (with 200 μl/mouse D-luciferase intraperitoneal injection), and pathology such as paralysis was monitored. As shown in FIG. 3A, tumor growth of KMS11-luciferase stably transduced with inducible NSD2 shRNA (sh4) greatly inhibited tumor growth after induction with doxycycline for 29 days. FIG. 3(B) shows a time course of luciferase signal from tumors formed by KMS11-sh4 with or without doxycycline induction. Luciferase activity was greatly reduced in the mice expressing NSD2 shRNA. FIG. 4 shows the percentage of paralyzed mice in groups of KMS11-sh4 with or without doxycycline induction. In conclusion, the NSD2 shRNA knockdown inhibited tumor initiation and prolonged a tumor and pathology-free period. The effect of shRNA knock down of NSD2 correlated both in tumorigenesis as the bottom 20% of mice that failed to develop tumors also did not develop any paralysis pathology.

### Example 4

### Clustering for NSD2 Mutations

Multiplexed library for exome capture sequencing was constructed utilizing the custom SureSelect Target Enrichment System (Agilient Technologies, Santa Clara, Calif.). Genomic DNA from cell lines was sheared and ligated to Illumina sequencing adapters including 8 bp indexes. Adaptor ligated DNA was then size-selected for lengths between 200-350 bp and hybridized with an excess of bait in solution phase.

Barcoded exon capture libraries were then pooled and sequenced on Illumina instruments (76 bp paired-end reads). The 8 bp index was read by the instrument at the beginning of read 2 and used to assign sequencing reads to a particular sample in the downstream data aggregation pipeline.

Sequence reads were aligned to NCBI Human Reference Genome GRCh37 by BWA software (Li et al., Bioinformatics 2010 25:1754-60) Sequence reads corresponding to genomic regions that may harbor small insertions or deletions (indels) were jointly realigned using GATK local realigner (DePristo et al., Nat Genet 2011 43:491-8) as described in to improve detection of indels and to decrease the number of false positive single nucleotide variations caused by misaligned reads, particularly at the 3′ end. Sites that are likely to contain indels were defined as sites of known germline indel variation from dbSNP database (Sherry et al., Nucleic Acids Res 2001 29:308-11) sites containing reads initially aligned by BWA with indels and sites adjacent to the cluster of detected nucleotide substitutions.

Variants Calling, Annotation and Filtering.

Nucleotide substitutions were detected with MuTect and short indels were called with Indelocator software developed at Broad Institute. Both programs were evoked in the mode that does not require matching normal DNA and identifies all variants that differ from the reference genome. Detected variants were annotated using reference transcripts derived from transcripts from the UCSC Genome Browser's “UCSC Genes” track.

Exclusion of Variants with Low Alleleic Fraction

Allelic fraction was calculated for each detected variant in each sample as a fraction of reads that support alternative (different from the reference) allele among reads overlapping the position. To limit the effects of potential sample contamination, sub-clonal events and false positives due-to alignment artifacts only mutations with allelic fraction above 20% were used in the downstream analysis.

Exclusion of Common Germline Variants

Variants that have been previously reported as germline polymorphism and for which global allele frequency (GAF) in dbSNP134 or allele frequency detected in the NHLBI Exome Sequencing Project was higher than 0.1% were excluded from further analysis. Natural selection is known to be very efficient at eliminating functional deleterious mutations and usually does not allow them to reach relatively high frequency in populations; however polymorphisms at the low end of population frequency can be extremely deleterious and be identical to some of the somatic mutations. Thus few mutations identical to known germline polymorphisms, but with population frequency at or below 0.1% were retained.

Exclusion of Variants Observed in Panel of Normals

Variants detected also in a panel of 278 whole exomes samples sequenced at the Broad as a part of 1000 Genomes Project were excluded from further analysis. In addition to removing additional germline variation this step allowed efficient removing of common false positives originating predominantly from the alignment artifacts.

Exclusion of Neutral Mutations

Any amino acid substitution that creates residue observed as a wild type at the homologous position in protein's orthologs in at least two warm blooded vertebrates, was excluded from further analysis as likely being neutral. For this filtering step we used multiple amino acid alignments created by BLASTZ program and obtained from University of California Santa Cruz, Genome Browser repository.

Identification of E1099K, an Activating Recurrent Mutation in NSD2

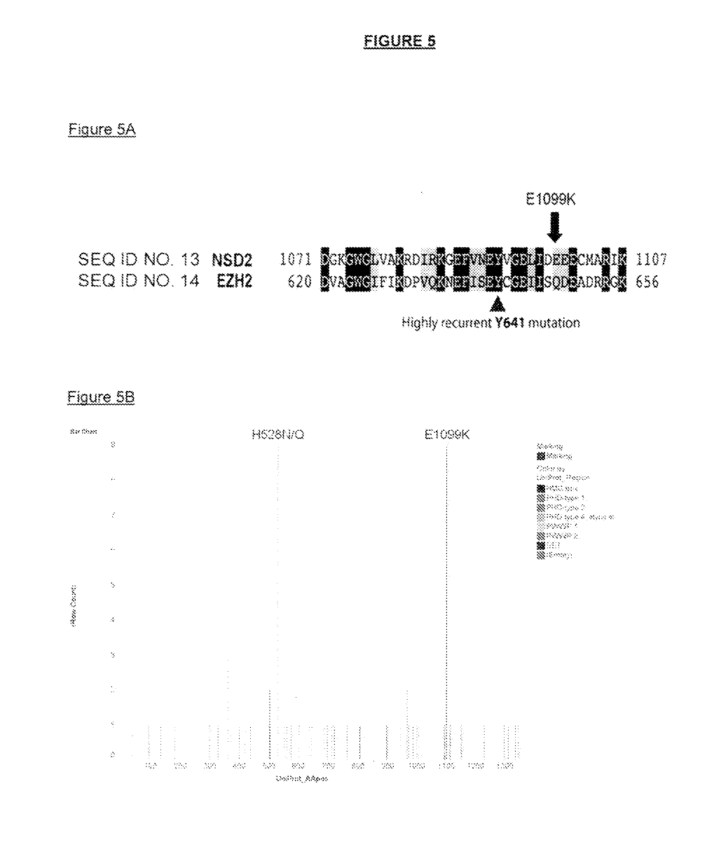

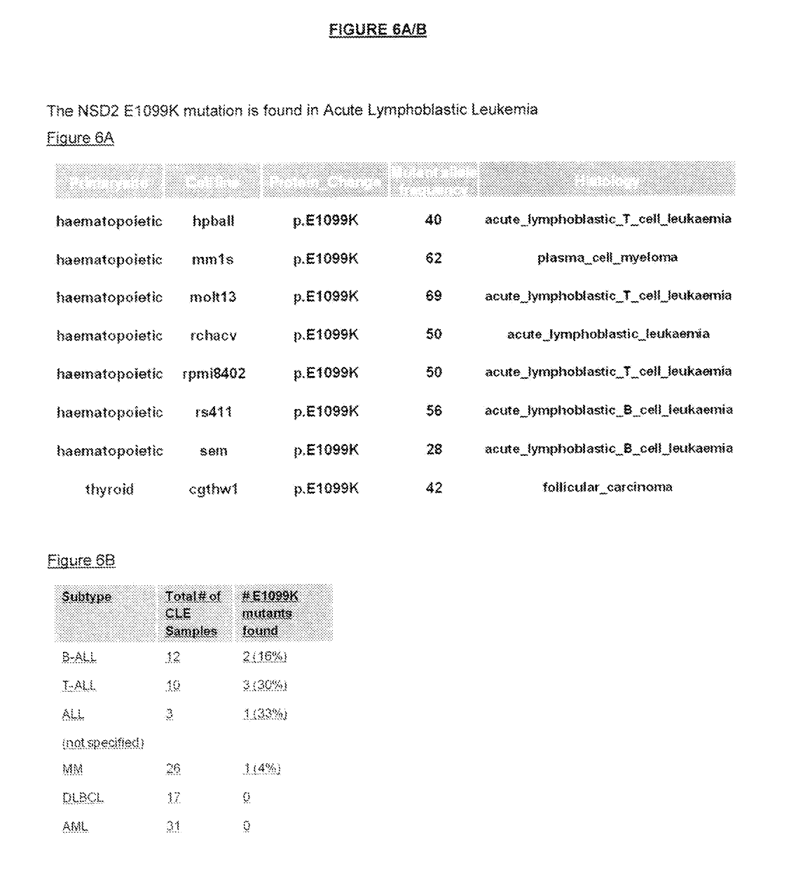

To identify recurrent oncogenic mutations we searched for missense variants absent in databases of germline SNPs, but preset in multiple cancer cell lines. To further filter out remaining rare germline polymorphisms, mutations found on a long haplotypes shared among mutated samples were excluded. The top remaining hit in this analysis was the E1099K mutation in NSD2 gene. This amino acid substitution was observed only in cell lines established from hematological and thyroid cancers. It is located in the NSD2 domain that is highly homologous to EZH2 histone methyltransferase catalytic domain. The affected E1099 amino acid residue is located next to the residue homologous to Y641 position in EZH2, which is a known hotspot for the recurrent activating mutations in hematological cancers (FIG. 5A). The position and frequency of the E1099K mutation in the full gene is shown graphically in FIG. 5B. FIG. 6A lists the type of ALL cells wherein the E1099K mutation would found, and FIG. 6B provides the number of mutations found in the respective cell types. The conclusion is that specific NSD2 mutations are found in cancers of lymphocytic origin, for example, ALL.

### Example 5

### Molecular Modeling

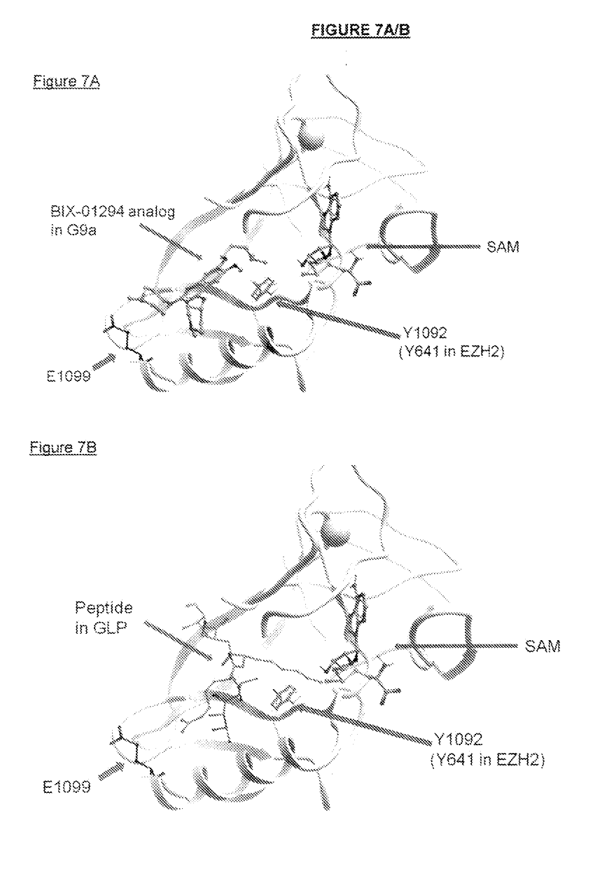

To understand the mutations in NSD2 and how it affects activity of NSD2, especially the E1099K mutation, a homology model of NSD2 was built. This model included other methyltransferases such as NSD1 (Protein data Bank code OOI) GLP/EHMT1 (Protein data Bank code 3HNA), G9a (Protein data Bank code 3k5k) and EZH2 (homology model built from MLL Protein data Bank code 2W5Z). All homology models were constructed using the 2010 version of Maestra software from Schrödinger (Cambridge, Mass.). FIG. 7A shows the positions of s-adenosylmethonine (SAM), BIX-01294 analog in G9a, E1099 (NSD2) and Y1062. FIG. 7B shows the positions of SAM, H3K9 peptide in GLP, E1099(NSD2) and Y1062. From the modeling alignments, E1099(NSD2) is located at substrate pocket of SET domain. In the GLP model, the corresponding amino acid to E1099(NSD2) is D1131, which has a salt bridge interaction with arginine near lysine (H3K9). Without being bound to any one hypothesis, it is likely that the E1099K mutation affects the binding of H3K36 to the NSD2 molecule, facilitating increased methyltransferase activity.

### Example 6

### NSD2 E1099K is a Gain of Function Mutation

In order to examine the impact of the NSD2 E1099K mutation on methyltransferase activity, 6×10⁵/well 293T/17 (ATCC) cells were seeded in 6-well plates. After overnight incubation, cells were co-transfected with equal amount of NSD2 WT or NSD2 E1099K mutant plasmid and H3-flag-luciferase construct (protein product ˜75 kDa) using Lipofectamine 2000 following the manufacturer's protocol (Life Technologies, Carlsbad Calif.). The NSD2 WT and NSD2 E1099K mutant were cloned into the p3xFlag-CMV7.1 vector and the H3-Luc was cloned into the pCDNA3 vector. 24 hours post-transfection, cells were harvested with SDS lysis buffer (1% SDS, 10 mM EDTA, 50 mM Tris.HCl pH 8.1) and boiled. Lysate was quantitated by BCA protein assay (Pierce, Rockford, Ill.) and resolved on NuPAGE Novex gel. Proteins were either wet transferred (for NSD2) or dry blotted with iBlot system (Invitrogen, Carlsbad, Calif.). Then nitrocellulose membranes were probed with different antibodies according to manufacturer's recommended procedures. The antibodies used are shown below in Table 1. The Novartis antibody was internally generated and has no catalogue number.

TABLE 1

| Antigen | Vendor | Cell# | Host |
|---|---|---|---|
| NSD2 | Abnova | H00007468-B01P | Mouse |
| H3K36m1 | Novartis | | Rabbit |
| H3K36m2 | CST | 2901 | Rabbit |
| H3K36m3 | CST | 4909 | Rabbit |
| H3K27m3 | CST | 9733 | Rabbit |
| H3 | CST | 4499 | Rabbit |

### Abnova (Taipei City, TW), Cell Signalling Technologies (CST) (Beverly, Mass.)).

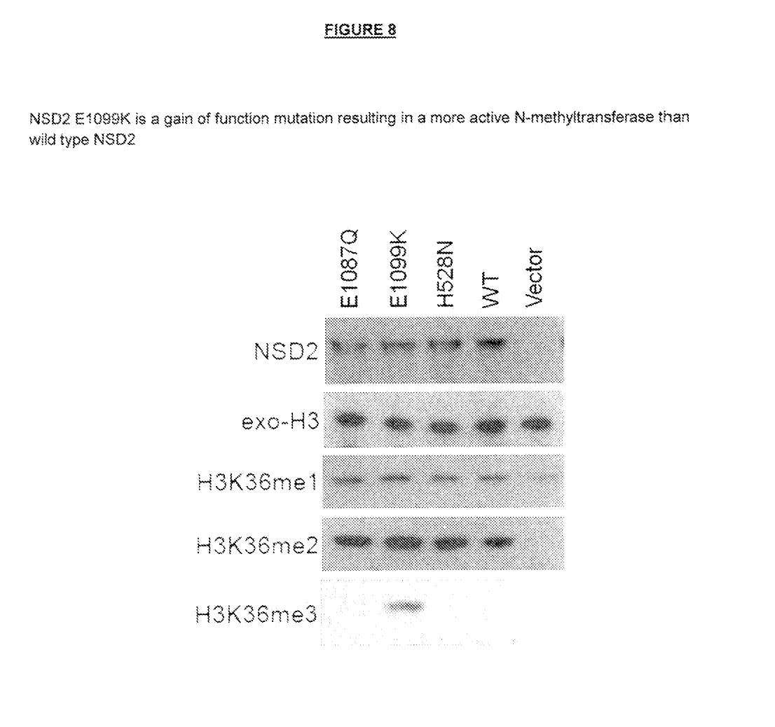

This experiment shows that NSD2 E1099K has increased N-methytransferase activity, as histone H3 at lysine 36, the known target for NSD2 is hypermethylated, with 3 methyl groups (me3) (see FIG. 8). In contrast, NSD2 WT activity produces only mono- to di-methylation at K36 (me1/me2). This indicates that the NSD2 E1099K mutation acts as a gain of function, which increases methyltransferase activity and can act to promote tumorigenesis.

### Example 7

### NSD2 E1099K Mutation has Increased Activity

In this experiment, NSD2 955-1365 wildtype or NSD2 E1099K mutant proteins were purified from E. Coli or mammalian (Flag tagged protein) systems and quantitated by densitometry. The enzymatic reactions (16 μL) were performed in white, 384-well proxiplate (Perkin Elmer, Waltham, Mass.) at room temperature for 2 hours in assay buffer (20 mM Tris-HCl, pH at 8.0, 0.01% Tween 20, 10 mM MgCl₂, 0.01% BSA and 1 mM DTT) containing indicated amount of enzyme, s-adenosylmethonine (SAM), and nucleosome. The reactions were stopped by the addition of 4 μL quench solution (2.5% TFA and 320 nM D4-SAH). The amount of S-adenosylhomocysteine (SAH) produced from the reactions was measured using an API4000 LC/MS/MS system (Absciex, Framingham, Mass.). D4-SAH was used as an internal standard (IS) for SAH-detection and normalization. SAH is an important predictor of cellular methylation potential and metabolic alterations associated with certain genetic or nutritional deficiencies (Melnyk et al., Clin. Chem. 2000; 46(2):265-272).

Liquid chromatography was performed on a Chromolith FastGradient HPLC column RP-18e, 25-2 mm (Merck, Whitehouse Station, N.J.). The column was connected to the mass spectrometer through a 6-port valve. The turbo ion electrospray was operated in the positive-ion mode. The precursor to product transitions for SAH (m/z 385.1→136.1) and D4-SAH (m/z 389.1→136.1) were monitored. Mobile phase A is 0.02% FA and 2% methanol in water, B is 0.1% FA in methanol. Injection volume was 4 μl and the autosampler was kept at 4° C. The eluents between 0.4 and 1.0 minute were diverted to mass spectrometer for analysis. The plot of SAH peak area/IS peak area vs SAH concentration was used to generate the normalization factor of SAH. The production of SAH from real enzymatic reaction was derived from the standard curve of SAH.

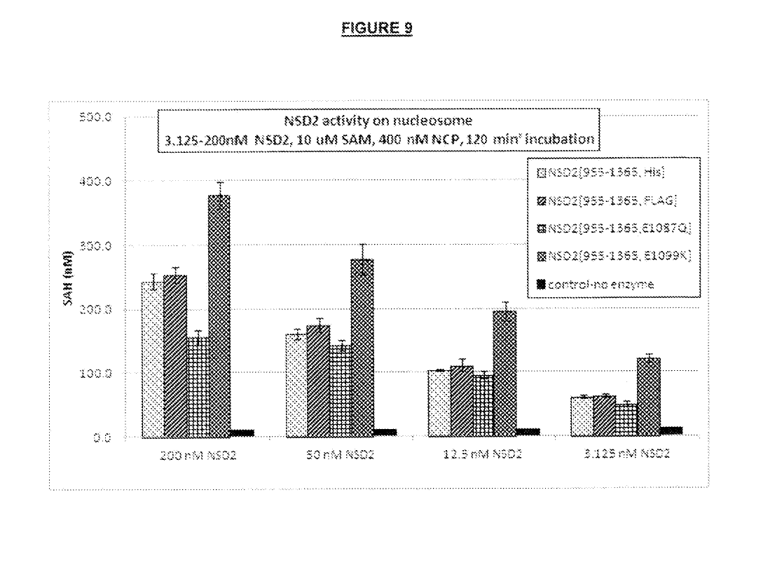

As can be seen in FIG. 9 the NSD2 E1099K protein gains extra activity on recombinant nucleosome compared to NSD2 WT. In contrast, the NSD2 E1087Q mutation has less N-methyltransferase activity in each case.

### Example 8

### NSD2 E1099K Shows Gain of Function in KO Rescued Lines

KMS11-TKO and corresponding parental lines were obtained from Horizon Discovery Ltd. (Cambridge, UK) and cultured in RPMI1640 (Gibco, Carlsbad, Calif.) supplemented with 12.5% fetal bovine serum (FBS, Gibco, Carlsbad, Calif.) and penicillin/streptomycin (Gibco, Carlsbad, Calif.). KMS11-TKO cells were generated by deletion of the exon 7 in the t(4,14) NSD2 translocated allele (Lauring, J. et al., 2008, Blood (111): 856-864). 293T/17 cells were purchased from ATCC and kept in DMEM (Gibco, Carlsbad, Calif.) with 10% FBS.

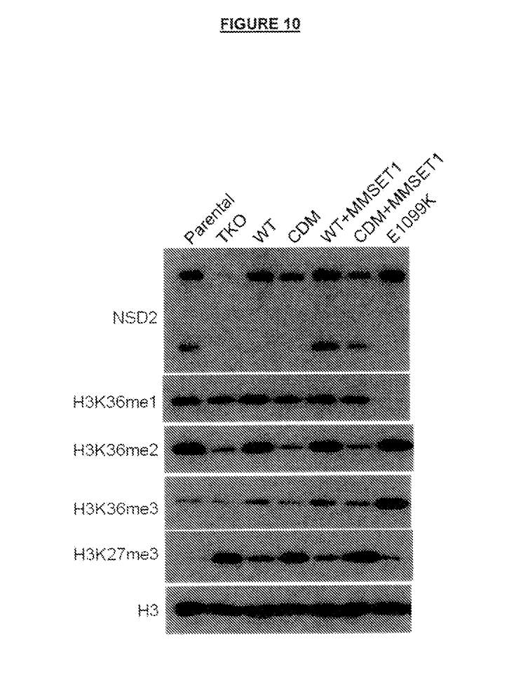

The catalytically dead NSD2 (CDM in FIG. 10) was generated as previously described in the literature (Kim, J. et al., Mol Cell Biol. 2008, (28):2023-2034).

Full-length NSD2 cDNAs (wildtype, catalytic dead mutant R1138A/C1144A, and E1099K) were cloned into pLenti6.3/blasticidin, and the N-terminal isoform MMSET1 was constructed into pLVXN/neomycin for lentiviral transduction. NSD2 mutants were generated by site-directed mutagenesis.

Lentiviral particles were produced by co-transfecting the above constructs with packaging plasmids Δ8.9 and VSVg into 293T/17 cells, using Lipofectamine LTX/PLUS reagents (Invitrogen, Carlsbad, Calif.) following the manufacturer's protocols. The KMS11 NSD2-TKO cells were infected with lentivirus in the presence of 8 μg/ml polybrene (Sigma, St. Louis, Mo.), and then selected and maintained under proper antibiotics (Gibco, Carlsbad, Calif.).

For Western blot analysis, cells were lysed with SDS lysis buffer (1% SDS, 10 mM EDTA, 50 mM Tris.HCl pH 8.1) and boiled. Lysate was quantitated by BCA protein assay (Pierce, Rockford, Ill.) and resolved on NuPAGE Novex gel. Proteins were either wet transferred or dry blotted with iBlot system (Invitrogen, Carlsbad, Calif.). Then nitrocellulose membranes were probed with different antibodies according to manufacturer's recommended procedures. The antibodies used are shown in Table 1 above.

As shown in FIG. 10, knockout of NSD2 on the translocated allele in t(4,14)+multiple myeloma cell line KMS11 caused dramatic decrease of H3K36me2 and increase of H3K27me3 (see TKO lane). Reconstitution of wild type (WT) but not catalytic dead mutant (CDM) full length NSD2, with or without the N-terminal isoform MMSET1, restored H3K36me2 and modestly increased H3K36me3 levels. Note that MMSET1 is a short isoform generated by alternative splicing of 647 amino acids which does not contain the SET domain (Lauring et al., supra). E1099K NSD2 further elevated H3K36me3 levels and reduced H3K36me1 compared to WT, indicating that the E1099K mutation results in a gain of function. The increased H3K36me2/3 by E1099K mutant NSD2 also antagonized methylation of histone H3 at amino acid 27 (lysine), resulting in a decrease of H3K27me3.

### Example 9

### Histone H3 Methylation of KMS11 Knockout Lines Reconstituted with the NSD2 E1099K Mutant

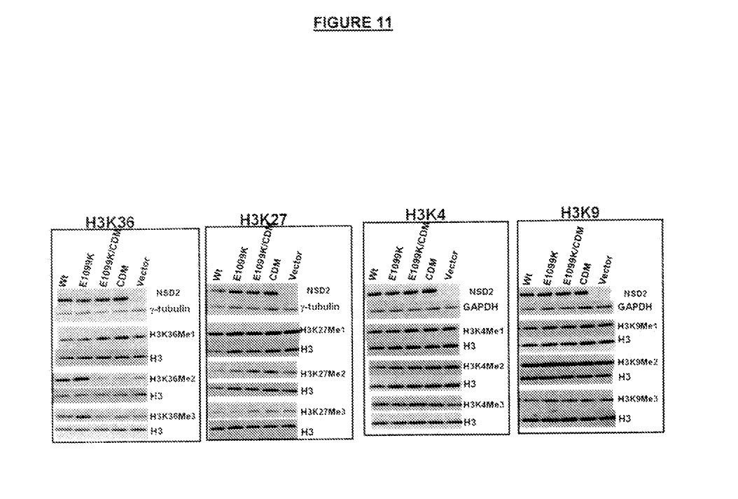

KMS11 knockout lines were propagated and maintained as described above. cDNA of NSD2 WT, NSD2 CDM or NSD2 E1099K were cloned into lenti protein overexpression vector pLVX-IRES-Neo (Clontech cat#632181, Mountain View, Calif.). In this Example and the accompanying Figures, CDM stands for a catalytically dead NSD2, as described above, and a clone was created where mutations to the catalytic site were engineered into the E1099K mutant. As shown in FIG. 11, “E1099K/CDM” denotes this double mutant construct., Packaging of lentivirus was achieved by transfecting pLVX-IRES-Neo vector containing cDNA with packaging vectors (VSVG, pMDL.gp.RRE & pRSV-REV) into 293T cells. KMS11-TKO cells were infected with lenti virus and selected by neomycin. The cell lines were cultured in RPMI 1640 (Hyclone, Cat#: SH30027.02, Logan, Utah) containing 10% FBS (Sigma, St. Louis Mo., Cat#: F6178-50 ml). Cell pellets for Western were harvested at the end of proliferation assay when cell counts are between 0.5-1 million/ml. Cell lysate was prepared by suspending pellet in loading buffer (58 mM Tris-HCl, 5% glycerol, 1.7% SDS, 0.1M DTT). Samples were heated at 95° C. for 3 minutes and run on NuPAGE Novex 4-12% Bis-Tris gel (cat#345-0124, Bio-Rad, Hercules, Calif.). Gel was wet transferred to Nitrocellulose membrane. Primary antibodies were used as previously described.

That NSD2 E1099K has increased methyltransferase activity was demonstrated by “reconstituting” experiments using KMS11-TKO cells. These cells transfected with full length NSD2 containing the E1099K mutation showed enhanced methyltransferase activity, resulting in higher levels of H3K36me2 and H3K36me3 than KMS11 TKO cells transfected with NSD2 WT. In the KMS-TKO background, both NSD2 WT and NSD2 E1099K showed dramatically higher levels of H3K36me2 than all CDM, with NSD2 E1099K demonstrating the highest level of H3K36me3. Conversely, both NSD2 WT and NSD2 E1099K showed lower levels of H3K36me1 than their respective CDMs and this most likely results from more efficient methylation from mono to di-methylation then tri-methylation by NSD2 WT and NSD2 E1099K mutant. Here, the experiments confirmed that high levels of H3K36 methylation antagonized levels H3K27me3 and that increased H3K36 methylation is the hall mark of NSD2 E1099K methyltransferase activity. As shown in FIG. 11, the increased activity of E1099K indicates that H3K36me2 or H3K36me2\me3 antagonized H3K27me3. This antagonizing effect of E1099K on H3K27me3 can be seen in FIG. 11, where the H3K27me3 level is lower than NSD2 WT. However, the normal enzymatic activity of NSD2 or the increased activity of E1099K does not affect the methylation at H3K4 and H3K9 sites. In summary, the gain of function of E1099K and its stronger methyltransferase activity on H3K36me2/me3 is driving the increased proliferation of ALL cells.

### Example 10

### The NSD2 E1099K Mutant Confers an Increase in Cell Proliferation

The amount of cell proliferation was assayed for using the TKO/reconstituted cell lines described in Example 9 above. The proliferation assay for TKO derived cell lines were set at 0.1 million/ml at 2% or 10% FBS in RPMI1640. After 4 days culture, cells were counted using Cell Viability Analyzer (Vi-Cell™ XR, Beckman Coulter Indianapolis, Ind.).

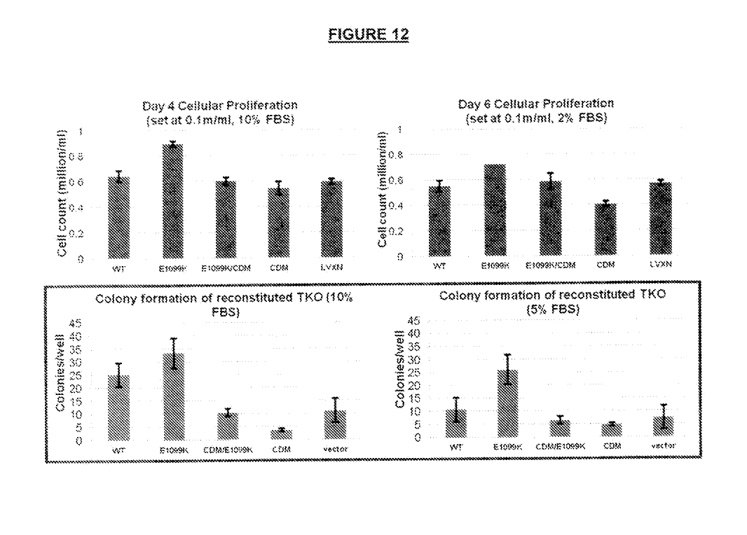

As shown in FIG. 12 top panel, the gain of function E1099K mutant confers a growth advantage over cells that do not contain the E1099K mutant.

Soft agarose colony formation assay were used to assess the tumorigenicity of TKO reconstituted with WT, E1099K and their respective CDM version. 0.3% agarose containing RPMI1640 & 5% or 10% FBS were used in 48 well plates. Four replicates for each sample were set up. Colony numbers were assessed in about 1.5 weeks.

As shown in FIG. 12 bottom panel, the gain of function E1099K mutation provides a colony formation growth advantage, so that greater numbers of colonies form in comparison with wild type or vector controls.

### Example 11

### Knockdown of NSD2 in ALL Cell Lines

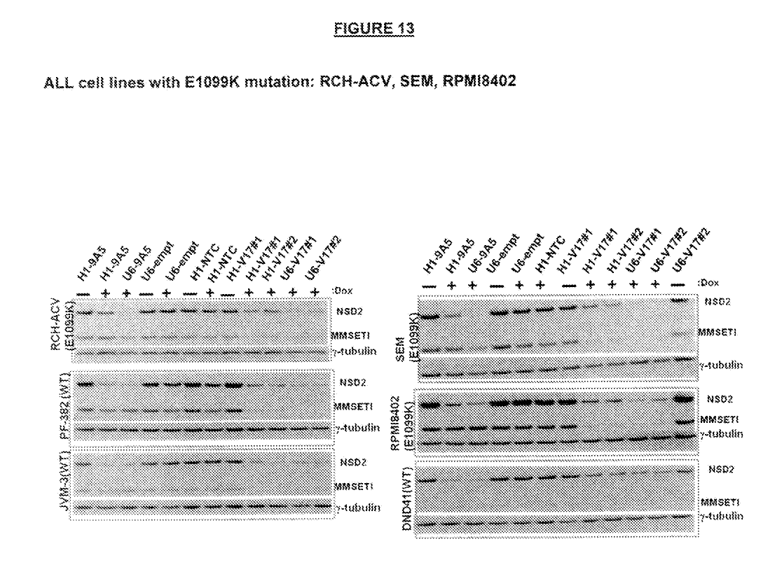

To test the hypothesis that the NSD2 E1099K mutation is important in the pathogenesis of ALL cells, several shRNA knockdown experiments were done. In these experiments, NSD2 shRNA sequences were cloned into pLKO-Tet-On vectors with the H1 promoter or U6 promoter. shRNA lentivitus was generated as described above. Cell lines were infected and selected by puromycin. All tet inducible shRNA cell lines are cultured using tet system approved FBS (Clontech, Cat#: 631106, Mountain View, Calif.). NSD2 Tet-inducible cellular proliferation assay was set up as the following. Cultured cells were placed at 0.2 million cells/ml in 10 ml and split into two wells (5 ml/well) in a 6-well plate. Doxycycline (100 ng/ml final) was added to one well at assay initiation and every 2-3 days to induce NSD2 Tet-inducible shRNAs. On days 4 and 8 the assayed cells (with and without doxycycline) were counted for viability and cell concentration (10⁶ cell/ml) with Vi-cell. Assayed cells were then reset to 0.2×10⁶ cells/ml in 5 ml every 4 days to keep the cells proliferating in log-phase. On day 8, 1 million cells were collected from assayed cells for Western blot analysis. The shRNA sequences used in the experiments were the following, and are used with this nomenclature in FIG. 13.

NSD2 shRNA V17: 5′ CGGAAAGCCAAGTTCACCTTT 3′ (SEQ ID NO. 9). This shRNA targets the NSD2 and MMSET1 isoforms.

NSD2 shRNA 9A5: 5′ GAATAAGCCTTACGGGAAA 3′ (SEQ ID NO. 10). This shRNA targets NSD2 isoforms containing the SET domain, such as the E1099K mutant.

Control shRNA sequence shGFP: 5′ GCTATAACTCACATAATGT 3′ (SEQ ID NO. 11). This is a non-specific shRNA directed against green fluorescence protein (GFP).

Control shRNA sequence: Empt: 8 bases non-specific sequence: 5′ AAGCTTTT 3′ (SEQ ID NO. 12). This is a control consisting of an 8 base pair scrambled sequence.

As can be seen from the Western blot results in FIG. 13, both the 9A5 shRNA and the V17 shRNA when induced with doxycycline were capable of reducing expression of NSD2 and MMSET1. For example, in the E1099K containing SEM cells, expression of NSD2 and MMSET1 were reduced to undetectable levels, especially when under control of the U6 promoter (FIG. 13, right panel).

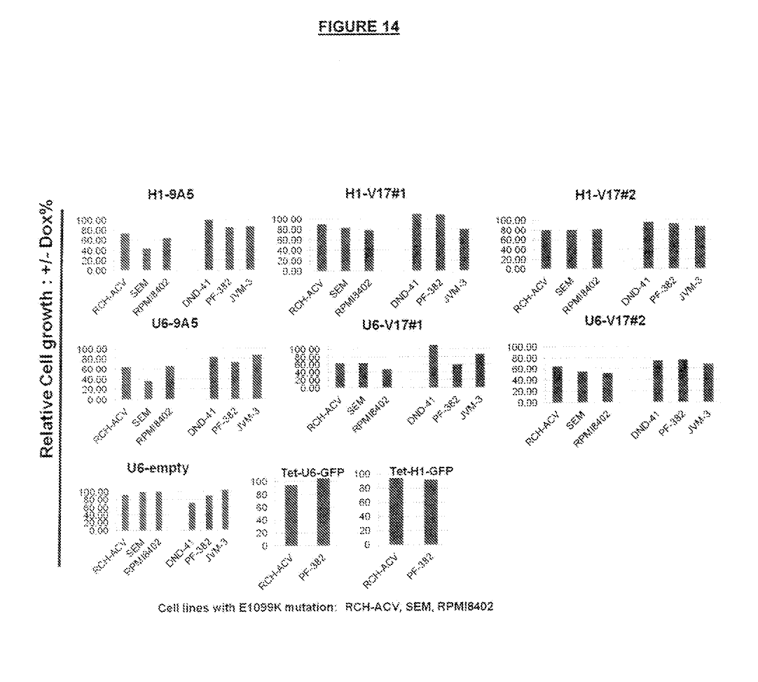

The shRNAs described above were then used in a cell proliferation assay. FIG. 14 shows the proliferation assay performed on the same ALL cell lines used for the evaluation of shRNA knockdown in FIG. 13. Note that the cell lines RCH-ACV, SEM and RPMI8402 are ALL cell lines containing the E1099K mutation. The DND-41, PF-382 and JVM-3 are ALL cell lines that do not contain the E1099K mutation. The proliferation of ALL cell lines containing the E1099K mutation were, in general, more effected by knockdown of NSD2. This can be seen in FIG. 14, for example, SEM cell proliferation was reduced by about 60%, when U6-9A5 shRNA was induced vs a U6-empty control. For the cell lines that do not contain the E1099K mutation, the effect of the shRNA knockdown was much less pronounced. For example, the proliferation of the JVM-3 line was reduced by only about 15% when U6-9A5 shRNA was induced vs a U6-empty control. In comparing the E1099K containing cells with the NSD2 WT containing cells, FIG. 14 concludes that for cells containing the E1099K mutation, a greater reduction in proliferation is seen when NSD2 is knocked down. As such, ALL cell lines containing the E1099K mutation are at least partially dependent upon the increased methyltransferase activity of the E1099K mutation for their increased growth.

### Example 13

### Methylation Status of E1099K ALL Cells Compared to Multiple Myeloma NSD2 t(4,14) Cells

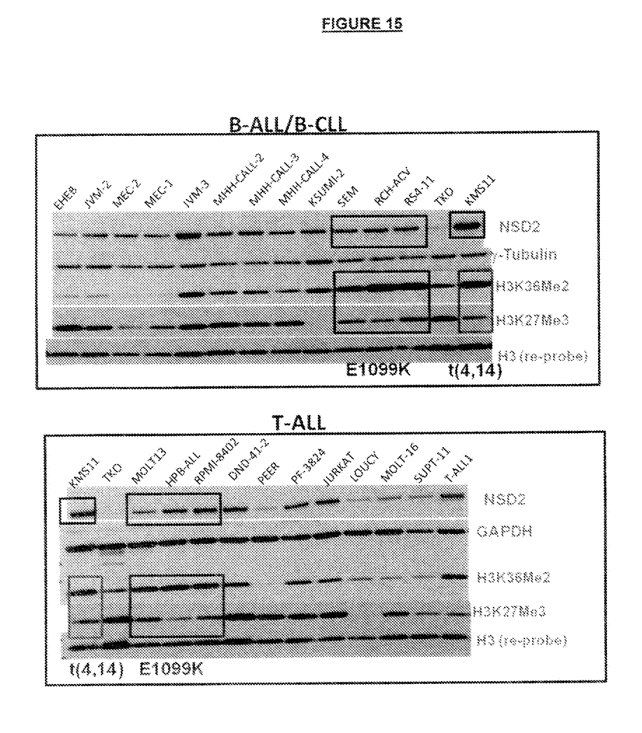

B-ALL, B-CLL (chronic lymphocytic leukemia), and T-ALL cell lines were harvested and Western blotting performed, comparing the histone methylation status with that of the multiple myeloma KMS11 line, which contains the NSD2 t(4,14) breakpoint mutation. Cell lines containing the NSD2 E1099K mutation, for example, SEM, RCH-ACV, RS4-11, MOLT13, HPB-ALL and RPMI-8402 were harvested and Western blotted as described above. The levels of histone methylation of H3K36me2 and H3K27me3 were compared to that of the KMS11 line. As shown in FIG. 15, H3K36me2 methylation is high in both the E1099K leukemic cells as well as the KMS11 cells. Similarly, H3K27me3 is low in both the E1099K leukemic cells and the KMS11 cells. This indicates that both mutations have a similar effect on H3 methylation and that ALL cells containing the E1099K mutation have tumorigenicity comparable to the known tumorigenic KMS11 multiple myeloma cells.

### Example 14

### Methylation Status of E1099K ALL Cells with shRNA Knockdown

NSD2 shRNA sequences were cloned into pLKO-Tet-On vectors with H1 promoter or U6 promoter. shRNA lentivitus was generated as described above. Cell lines were infected and selected by puromycin. All tet inducible shRNA cell lines are cultured using tet system approved FBS (Clontech, Cat#: 631106). The NSD2 Tet-inducible cellular proliferation assay was set up as follows. Cultured cells were placed at 0.2 million cells/ml in 10 ml and split into two wells (5 ml/well) in 6-well plate. Doxycycline (100 ng/ml final) was added to one well at assay initiation and every 2-3 days to induce NSD2 Tet-inducible shRNAs. On days 4 and 8 the assayed cells (with and without doxycycline) were counted for viability and cell concentration (10⁶ cell/ml) with Vi-cell. Assayed cells were then reset to 0.2×10⁶ cells/ml in 5 ml every 4 days to keep the cells proliferating in log-phase. On days 8, 1 million cells were collected from assayed cells for Western blot analysis. The shRNA sequences used in the experiments were the V17 (SEQ ID NO. 9) and 9A5 (SEQ ID NO. 10) as described in Example 11 above.

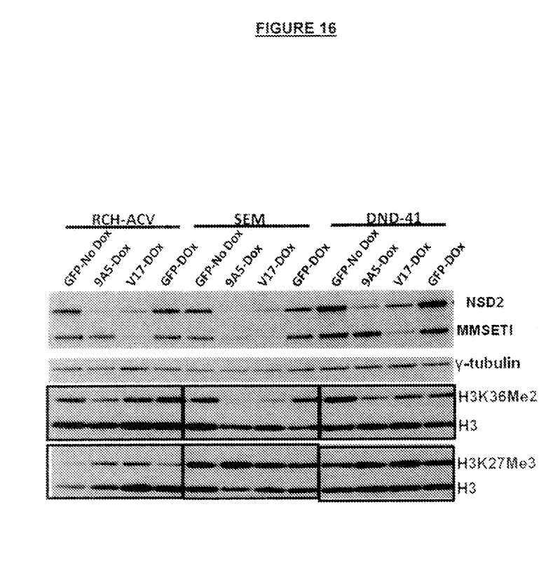

As shown in FIG. 16, shRNA reduction of NSD2 results in reduction of H3K36me2 methylation and an increase in H3K27me3 methylation. For example, this is clearly seen in SEM cells wherein NSD2 E1099K is knocked down with A95. The amount of NSD2 present is very low, as is H3K36me2 methylation. In contrast, the same SEM 9A5 lane in the Western blot shows robust H3K27me3 methylation.

### Example 15

### Growth of E1099K ALL Cell Lines in Soft Agar

Soft agarose colony formation assay were used to assess the tumorigenicity of T-ALL (8 lines), B-ALL (8 lines) and B-CLL (5 lines). 1500 and 750 cells were placed in 24 well plate with 0.2% agarose (Lonza, Cat#: 50101, Basel, Switzerland) containing RPMI1640 and 15% FBS. Colony formation was checked after one week.

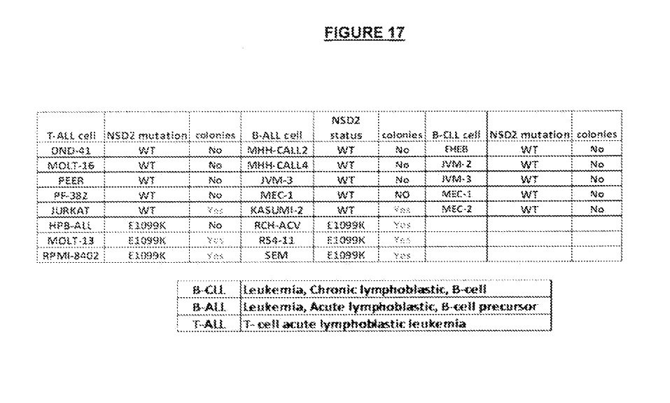

The result of this assay was that 5 out 6 ALL cell lines containing the E1099K mutation grow in soft agar and form colonies. In contrast, 2 out of 15 cell lines containing NSD2 WT were able to grow in soft agar and form colonies. This result is shown in FIG. 17 indicating that the presence of the E1099K mutation in NSD2 has a high correlation with tumorigenicity.

### Example 16

### Histone Profiling of Multiple Myeloma and ALL Cells

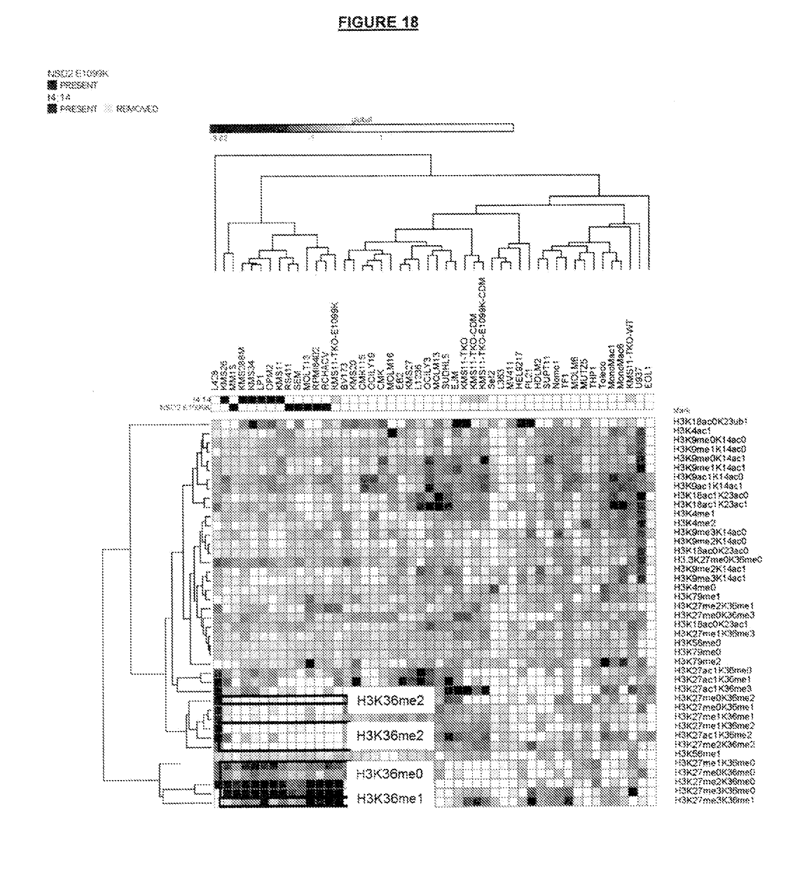

Histones were extracted from the selected cell lines or engineered derivative cell lines as previously described (Thomas et al., J. Prot. Res. 2006 5, 240-247). In parallel, histones were extracted from 293T, K562, and HeLa cell lines that had been cultivated using SILAC (Ong et al., Mol. Cell. Prot. 2002 1, 376-386). A standardization mixture consisting of equal parts of the SILAC histones from the 3 aforementioned cell lines was formulated. In turn, equal parts of the standardization mixture and histones from each selected cell line or engineered derivative cell line were formulated to constitute each “sample.” Histone peptides were prepared from each sample as previously described (Garcia et al., Nat. Protoc. 2007 2, 933-938) except NHS-propionate was used as the amine derivatization reagent and methyl esterification was omitted. Peptide samples were analyzed using liquid chromatography-mass spectrometry (LCMS) with a Q-Exactive hybrid quadrupole-electrostatic trap mass spectrometer using an acquisition method designed to isolate each peptide species of interest and cause it to be dissociated by collisional activation into fragment ions. Each peptide species of interest was quantified using Skyline software (MacLean et al., Bioinfo. 2010 26, 966-968) wherein the LCMS peak area ratios of the intensities of the fragment ions of each peptide in the selected cell line and the standardization mix were determined. These ratios were further normalized in each sample to the total amount of histones present in the cell line-derived sample and the standardization mix as determined by LCMS. To facilitate ease of display across the many samples, each peptide species of interest was normalized to the median of that species in all of the samples analyzed. Multiple histone H3 peptides carrying H3K36me2 modifications (including H3K27me(0-2), H3K27me2 and H3K36me2) are significantly enriched, and histone H3 peptides with H3K36me0 and H3K36me1 modifications (H3K27me(0-3), H3K36me0 and H3K27me3, H3K36me1) are significantly repressed in cell lines expressing NSD2 E1099K mutants, similar to the examples when NSD2 is overtly over-expressed by t(4;14) translocation. This is shown in FIG. 18, which displays the results of this experiment as a heat map. It is important to note the first two rows underneath each cell line indicating the t(4;14) status, either present or removed, and the NSD2 E1099K status, present or removed. NSD2 removal is indicated by a grey box, while the presence of the NSD2 mutation is indicated with a black box.

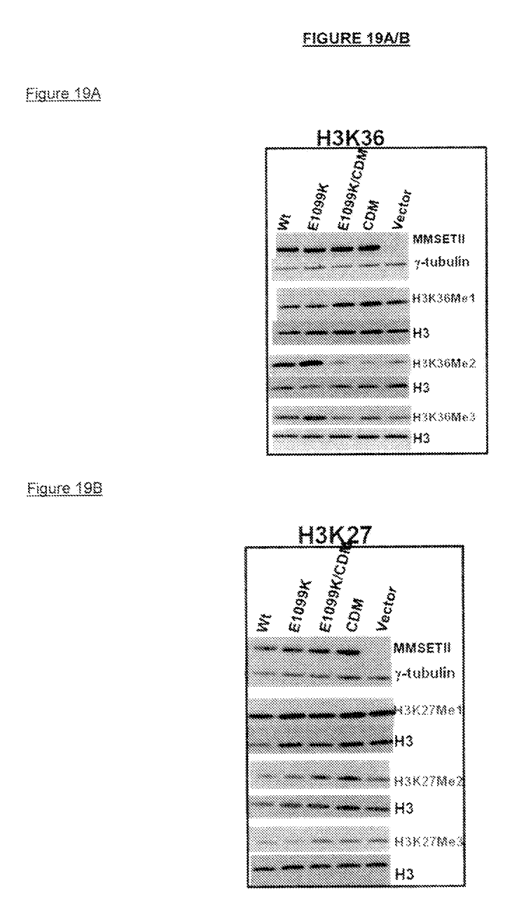

The mass spec result is confirmed by Western blotting as shown in FIGS. 19A/B. Westerns were performed using the protocol used previously (see above). The Western in FIG. 19A shows the increase in H3K36me2/me3 by the NSD2 E1099K mutant form, in contrast to the low amounts found in the CDM form. The converse is also true. The levels of H3K27me2/me3 are quite low in NSD2 E1099K containing lysates, when compared with the higher levels found with the CDM form.

1. A method of detecting a cancer cell, the method comprising;
a) obtaining a cancer sample from a patient;
b) screening for the presence of a Nuclear SET domain-containing protein (NSD2) mutation; and
c) comparing the NSD2 mutation to wild-type NSD2 in a non-cancerous or normal patient sample.

2. The method of claim 1, wherein the NSD2 mutation is a glutamic acid to lysine change at amino acid position 1099 (E1099K).

3. The method of claim 1, wherein the cancer cell is a leukemia cell.

4. The method of claim 3, wherein the leukemia cell is selected from the group consisting of: acute lymphoblastic T cell leukemia, acute lymphoblastic B cell leukemia, plasma cell myeloma, diffuse large B cell lymphoma and follicular carcinoma

5. A method of diagnosing cancer, the method comprising;
a) obtaining a cancer sample from a patient;
b) screening for the presence of a NSD mutation;
c) comparing the NSD2 mutation to wild-type NSD2; and
d) comparing the methylation of histone H3 at lysine 36 (H3K36) in the cancer sample with a NSD2 mutation, with the methylation of histone H3 at lysine 36 (H3K36) of a non-cancerous or normal patient sample, and increased methylation at H3K36 is indicative of cancer.

6. The method of claim 5, wherein the NSD2 mutation is E1099K.

7. The method of claim 5, wherein the cancer sample is leukemia.

8. The method of claim 7, wherein the leukemia sample is selected from the group consisting of: acute lymphoblastic T cell leukemia, acute lymphoblastic B cell leukemia, plasma cell myeloma, diffuse large B cell lymphoma and follicular carcinoma

9. The method of claim 5, wherein H3K36 is mono-methylated (H3K36me1), di-methylated (H3K36me2) or tri-methylated (H3K36me3).

10. The method of claim 5, further comprising comparing the methylation of histone H3 at lysine 27 (H3K27) in the cancer sample with an NSD2 mutation, with the methylation of histone H3 at lysine 27 (H3K27) of a non-cancerous or normal patient sample, and decreased methylation at H3K27 is indicative of cancer.

11. The method of claim 10, wherein H3K27 is unmethylated (H3K27me0), mono-methylated (H3K27me1), di-methylated (H3K27me2) or tri-methylated (H3K27me3).

12. A method of screening for a NSD2 inhibitor candidate, the method comprising:
a) contacting a cell containing a NSD2 mutation with a NSD2 inhibitor candidate;
b) measuring the methyltransferase activity; and
c) comparing the reduction in methyltransferase activity from the NSD2 mutant cell contacted with the NSD2 inhibitor candidate with methyltransferase activity of the methyltransferase activity of a normal or control cell and/or untreated cells containing the NSD2 mutation.

13. The method of claim 12, wherein the NSD2 mutation is E1099K.

14. The method of claim 12, wherein the cell containing a NSD2 mutation is selected from the group consisting of acute lymphoblastic T cell leukemia, acute lymphoblastic B cell leukemia, plasma cell myeloma, diffuse large B cell lymphoma, and follicular carcinoma.

15. Composition comprising a NSD2 mutation for use in diagnosis of cancer in a selected cancer patient population, wherein the cancer patient population is selected on the basis of containing a NSD2 E1099K mutation in a cancer cell sample obtained from said patients compared to a normal control cell sample.

16. The composition wherein the cancer sample is selected from the group consisting of is selected from the group consisting of: acute lymphoblastic T cell leukemia, acute lymphoblastic B cell leukemia, plasma cell myeloma, diffuse large B cell lymphoma, and follicular carcinoma.

17. A kit for predicting the sensitivity of a cancer patient for treatment with a NSD2 inhibitor comprising: i) means for detecting NSD2 E1099K mutation; and ii) instructions how to use said kit.


SEQUENCE LISTING (14 sequences)

SEQ ID NO: 1 - DNA, 7418 bp, Homo sapiens
tgttctaagaacggaagcatctgggctggatggaatttagcatcaagcagagtcccctttctgttcagagtgttgtaaagtgcataaagatgaagcaggcaccagaaatcctcggcagtgccaacgggaagactccgagctgcgaggtgaaccgcgagtgttctgtgttcctcagcaaagcccagctctccagtagcctgcaggagggggtcatgcagaagtttaacggccacgacgccctgccctttattccagccgacaagctgaaagatcttacttcccgggtgtttaatggagaacccggcgcacacgatgccaaactgcgttttgagtcccaggaaatgaaagggattgggacaccccctaacactacccctatcaaaaatggctctccagaaattaagctgaaaatcaccaaaacatacatgaatgggaagcctctctttgaatcttccatttgtggtgacagtgctgctgatgtgtctcagtcagaagaaaatggacaaaaaccagaaaacaaggcgagaaggaacaggaagaggagcataaaatatgactccttgctggagcagggccttgtcgaagcagctcttgtgtctaagatctcaagtccttcagataaaaagattccagctaagaaagagtcttgtccaaacactggaagagacaaagaccacctgttgaaatacaacgttggtgatttggtgtggtccaaagtgtcgggttacccttggtggccttgcatggtttctgcagatccactccttcacagctataccaaacttaaaggtcagaaaaagagtgcacgccagtatcacgtacagttctttggtgacgccccagaaagagcttggatatttgagaagagcctcgtagcttttgaaggagaaggacagtttgaaaaattatgccaggaaagtgccaagcaggcacccacgaaagctgagaaaattaagctattgaaaccaatttcagggaaattgagggcccagtgggaaatgggcattgttcaagcagaagaagctgcaagcatgtcagtggaggagcggaaagccaagttcacctttctctatgtgggggaccagcttcatctcaaccctcaagtagccaaggaggctggcattgctgcagagtctttgggagaaatggcagaatcctcaggagtcagtgaagaagctgctgaaaaccccaagtctgtgagagaagagtgcattcccatgaagagaaggcggagggccaaactgtgtagctctgcagagaccctggagagtcaccccgacatagggaagagtactcctcaaaagacggcagaggctgaccccagaagaggagtagggtctcctcctgggaggaagaagaccacagtctccatgccacgaagcaggaagggagatgcagcatcccagtttttggtcttctgtcaaaaacacagggatgaggtggtagctgagcacccagatgcttcaggtgaggagattgaagagctgctcaggtcacagtggagtctgctgagtgagaagcagagagcacgctacaacaccaagtttgccctggtggcccctgtccaggctgaagaagactctggtaatgtaaatgggaaaaaaagaaaccacacaaagaggatacaggaccctacagaagatgctgaagctgaggacacacccaggaaaagactcaggacggacaagcacagtcttcggaagagagacacaatcactgacaaaacggccagaacaagctcttacaaggccatggaggcagcctcctcgctcaagagccaggcagcaacgaaaaatctgtctgatgcatgtaaaccactgaagaagcgaaatcgggcttccacggcagcatcttcagctcttgggtttagcaaaagttcatctccttctgcatccttaactgagaatgaggtctcggacagcccgggagacgagccctcggagtccccatacgaaagtgcagacgaaacacaaactgaagtatctgtctcatccaaaaagtctgagcgaggagtgactgccaaaaaggagtatgtgtgccagctgtgtgagaagccgggcagcctcctgctctgtgaaggaccctgctgcggagctttccacctcgcctgccttgggctttcccggaggccagaagggaggttcacctgcagcgagtgtgcctcagggattcactcatgtttcgtgtgtaaagagagcaagacagatgttaagcgctgtgtggtaactcagtgtggaaaattttaccatgaggcttgtgtgaaaaaataccctctgactgtatttgagagccgaggtttccgctgccccctccacagctgtgtgagctgccatgcttccaacccttcaaacccaaggccgtcaaaaggtaaaatgatgcggtgtgtccgctgccccgttgcctatcacagcggggatgcttgtctggcagcaggatgctcagtgatcgcctccaacagcatcatctgcactgcccacttcactgctcggaaggggaagcgacaccacgcccacgtcaacgtgagctggtgcttcgtgtgctccaaaggggggagccttctgtgctgtgagtcctgcccagcggccttccaccctgactgcctgaacatcgagatgcctgacggcagctggttctgcaatgactgcagggctgggaagaagctgcacttccaggatatcatttgggtgaaacttgggaactacagatggtggccggcagaagtttgccatcccaaaaatgttcccccaaatattcagaaaatgaagcacgagattggagaattccctgtgtttttctttgggtctaaagattattactggacgcatcaggcgcgagtgttcccgtacatggagggggaccggggcagccgctaccagggggtcagagggatcggaagagtcttcaaaaacgcactgcaagaagctgaagctcgttttcgtgaaattaagcttcagagggaagcccgagaaacacaggagagcgagcgcaagcccccaccatacaagcacatcaaggtgaataagccttacgggaaagtccagatctacacagcggatatttcagaaatccctaagtgcaactgcaagcccacagatgagaatccttgtggctttgattcggagtgtctgaacaggatgctgatgtttgagtgccacccgcaggtgtgtcccgcgggcgagttctgccagaaccagtgcttcaccaagcgccagtacccagagaccaagatcatcaagacagatggcaaagggtggggcctggtcgccaagagggacatcagaaagggagaatttgttaacgagtacgttggggagctgatcgacgaggaggagtgcatggcgagaatcaagcacgcacacgagaacgacatcacccacttctacatgctcactatagacaaggaccgtataatagacgctggccccaaaggaaactactctcgatttatgaatcacagctgccagcccaactgtgagaccctcaagtggacagtgaatggggacactcgtgtgggcctgtttgccgtctgtgacattcctgcagggacggagctgacttttaactacaacctcgattgtctgggcaatgaaaaaacggtctgccggtgtggagcctccaattgcagtggattcctcggggatagaccaaagacctcgacgaccctttcatcagaggaaaagggcaaaaagaccaagaagaaaacgaggcggcgcagagcaaaaggggaagggaagaggcagtcagaggacgagtgcttccgctgcggtgatggcgggcagctggtgctgtgtgaccgcaagttctgcaccaaggcctaccacctgtcctgcctgggccttggcaagcggcccttcgggaagtgggaatgtccttggcatcattgtgacgtgtgtggcaaaccttcgacttcattttgccacctctgccccaattcgttctgtaaggagcaccaggacgggacagccttcagctgcaccccggacgggcggtcctactgctgtgagcatgacttaggggcggcatcggtcagaagcaccaagactgagaagccccccccagagccagggaagccgaaggggaagaggcggcggcggaggggctggcggagagtcacagagggcaaatagcgccaggcggccgcttggccggatccaggggcggtgcagggcggccggccctgcctgcgggagagggcgagcatgaactggcccggaggacccagctcgagccgccaggacacagacgtacaggcctcctcgggagggagcgcctccccaccactgagccatcctcagcagcgtccgctgcgtctgcactgatgaccgtctgagcccagctcagcgttcctggacaaacagcctcactcctcagcgttaccgccacacttgaatttctccgaatgtcaaggttccctcccactctatttttttaggttaaagttaattggcatatggaatgttttaatctcctctgaaatgtgtagcgtaggcttttcccaagggtcgctagaaactcgtcttcgcgttgccccctttctggctctcagcgccgtcgccactcgggagaggctgggtgaggcccgtgtgaggactgaccctggattcctcgaaactgccattgtgatcattactctgctctttggaaatggctgtatcatttttttgtactaatgtgaattgttcctcagaaacgcttcttttccatcctagtgagaagctggccctgcaggtggtggcagcaatggtgttgtaagatttcctcccgtagttttttctcctcatggatttgaatgaaatgccaataacacgtccactttcaacgtgtagtttacgcggagcactttcgaggcctggccgggttgggcctacttctcacctgggcctatcttctgaactcgctaggttcttatcaacatttgggggataactttgtatatttttttcatttggcttttctttaccagtttctgatttttattctcaatatatttttgctaaacctatttcacaaatcaccaccgactgaagtgtgtgtttactgatgcggccctgagctccatggcgaaaggagtgactttgcagggcgtgagaccgcagtctgcttagagcacaggaagtgacaacttagggagccccgtagggcgctgcaggccccggggaccccagcacgtgggtctaaagagagacggagtctagctctcctgccacccagagtggcttccatctcagcactctgtgggtctggtgatggaagatgcagtctctgctgatcacatgtgccctctgccagggcacctactgagaggtgcggtcctgggggtggaggcctgcctggcaggtgtgcgtgcctcgtacgtgtgttatgggcactggtctaggccaggtatgacacccactctcctgtgagatttcactttagtttttaaaaggtccagttctacagagtgagacctatctatctgagtactacatatgttttaagacttggttctttttttgagggatccttgaccctgggaagtctggagcaccctgagaagggggcaccatgtgtgcctttgcccacgtgtcctgaggggctgcttgtctgggagggagggagagaacattcagcagcaggtgcttttttatggccttttcttaaaataacctaagggggacacatccatcttgcagagaagtttacagaactccccttgaaaactgctgctgaggctcctgttaaattttctgtggcatcttttatgccttggtaaaaactgcagtgtctttggacctgagagtggctactccgtggttttgtgacctgtaagcgtggggttcaggggtgtgtggccctgcagggtcccacgcctccctgagcactgactggaagtttcactggctggtggctgtcccttctcccatcagggtccccagcaaagttaactacacagaggacccaggggaaacgagctgtgtagccactgacttgctcgcgcggccgtggcctctgaggggcactcgccggttaagacagggtgggagtagtgctttccagttcagactctaacttctcccaaagtgtcctaagaaaatactggatcggctcatagatttatgctccttatgatgccctaacttggaaggttgttctagggacaggccgggcagtgtccccacacacaccttagagtcgaaggccccagggccccgctgtcacttgcccaaaagatcccttccggcaggtaagggactaccaatgcttacgtcaaaacagcagaatcggctttgcagtgcactttggggagcagatattaacttatttttgtgttggacagtagtgaaatcttgtgatttttaatcgctttgataatacttccaaattttatgatttttctgaaggaaataatgcaaacattttaaatatgtttctccccctttccaaaaactgttaaactaatgagcaagtaacactaactttgaatgtctctacaatacccgttgataactcagtggagccaggctttggggtagcggccctgagcttgcagggtttctcgccactggggctgaccacgcccccagctgtgaccgtgggtgtggctggctctcggccctgcccagctttgttctgaggacgtggtgacttcctgaacatcagcttcaatcctccatcattaatgtgaagcaaaacacaaaaaccgccccaatccctcaggattccttggcatccgaaaccagcatctgcacctaaacccatacccacccgtgtgcgcccacagggggatgtgtccgaatgggcagcttaaaatgtggtcacctgtgggggaaactcttcaggcacctgaagtgagaacccagctgtccgtcctcaggccggcctttcttccggcgacacccgtccatggctggctgggtccccttcgcagtgtttgtctgtcttgacatctaaaccccggcgtgtgcagtgcccatcttccaggactaccttattttccagaattaaacctgttttataattcaagttaatgcaaatgactgtcagttgccaaatatcttgatcctgtgagtgtagttgatgactgtttgttagtcagtagagtaaaatgctgtgtccacggggtgtcacagcctcaccataccctgttgaggtgtgaaatgccccgtcagaaattaaatacaaacttaaatgtgcctattggtgtctaaacttcatacaatgtaaggtcagattccttttaggaatactgggtgctgtcaccaggtttgatagttagacttaaaaacttgaaattcactttttggggggagggatatactgaaatagagagttgagacttgccagttgggggaaaatagcatttaaaatggaaagctgtgtttggaaaattgtgtatgagtatttttgtattaaaaacattttaaaggcttttttct

SEQ ID NO: 2 - protein, 1365 aa, Homo sapiens
MEFSIKQSPLSVQSVVKCIKMKQAPEILGSANGKTPSCEVNRECSVFLSKAQLSSSLQEGVMQKFNGHDALPFIPADKLKDLTSRVFNGEPGAHDAKLRFESQEMKGIGTPPNTTPIKNGSPEIKLKITKTYMNGKPLFESSICGDSAADVSQSEENGQKPENKARRNRKRSIKYDSLLEQGLVEAALVSKISSPSDKKIPAKKESCPNTGRDKDHLLKYNVGDLVWSKVSGYPWWPCMVSADPLLHSYTKLKGQKKSARQYHVQFFGDAPERAWIFEKSLVAFEGEGQFEKLCQESAKQAPTKAEKIKLLKPISGKLRAQWEMGIVQAEEAASMSVEERKAKFTFLYVGDQLHLNPQVAKEAGIAAESLGEMAESSGVSEEAAENPKSVREECIPMKRRRRAKLCSSAETLESHPDIGKSTPQKTAEADPRRGVGSPPGRKKTTVSMPRSRKGDAASQFLVFCQKHRDEVVAEHPDASGEEIEELLRSQWSLLSEKQRARYNTKFALVAPVQAEEDSGNVNGKKRNHTKRIQDPTEDAEAEDTPRKRLRTDKHSLRKRDTITDKTARTSSYKAMEAASSLKSQAATKNLSDACKPLKKRNRASTAASSALGFSKSSSPSASLTENEVSDSPGDEPSESPYESADETQTEVSVSSKKSERGVTAKKEYVCQLCEKPGSLLLCEGPCCGAFHLACLGLSRRPEGRFTCSECASGIHSCFVCKESKTDVKRCVVTQCGKFYHEACVKKYPLTVFESRGFRCPLHSCVSCHASNPSNPRPSKGKMMRCVRCPVAYHSGDACLAAGCSVIASNSIICTAHFTARKGKRHHAHVNVSWCFVCSKGGSLLCCESCPAAFHPDCLNIEMPDGSWFCNDCRAGKKLHFQDIIWVKLGNYRWWPAEVCHPKNVPPNIQKMKHEIGEFPVFFFGSKDYYWTHQARVFPYMEGDRGSRYQGVRGIGRVFKNALQEAEARFREIKLQREARETQESERKPPPYKHIKVNKPYGKVQIYTADISEIPKCNCKPTDENPCGFDSECLNRMLMFECHPQVCPAGEFCQNQCFTKRQYPETKIIKTDGKGWGLVAKRDIRKGEFVNEYVGELIDEEECMARIKHAHENDITHFYMLTIDKDRIIDAGPKGNYSRFMNHSCQPNCETLKWTVNGDTRVGLFAVCDIPAGTELTFNYNLDCLGNEKTVCRCGASNCSGFLGDRPKTSTTLSSEEKGKKTKKKTRRRRAKGEGKRQSEDECFRCGDGGQLVLCDRKFCTKAYHLSCLGLGKRPFGKWECPWHHCDVCGKPSTSFCHLCPNSFCKEHQDGTAFSCTPDGRSYCCEHDLGAASVRSTKTEKPPPEPGKPKGKRRRRRGWRRVTEGK

SEQ ID NO: 3 - protein, 1365 aa, Homo sapiens
MEFSIKQSPLSVQSVVKCIKMKQAPEILGSANGKTPSCEVNRECSVFLSKAQLSSSLQEGVMQKFNGHDALPFIPADKLKDLTSRVFNGEPGAHDAKLRFESQEMKGIGTPPNTTPIKNGSPEIKLKITKTYMNGKPLFESSICGDSAADVSQSEENGQKPENKARRNRKRSIKYDSLLEQGLVEAALVSKISSPSDKKIPAKKESCPNTGRDKDHLLKYNVGDLVWSKVSGYPWWPCMVSADPLLHSYTKLKGQKKSARQYHVQFFGDAPERAWIFEKSLVAFEGEGQFEKLCQESAKQAPTKAEKIKLLKPISGKLRAQWEMGIVQAEEAASMSVEERKAKFTFLYVGDQLHLNPQVAKEAGIAAESLGEMAESSGVSEEAAENPKSVREECIPMKRRRRAKLCSSAETLESHPDIGKSTPQKTAEADPRRGVGSPPGRKKTTVSMPRSRKGDAASQFLVFCQKHRDEVVAEHPDASGEEIEELLRSQWSLLSEKQRARYNTKFALVAPVQAEEDSGNVNGKKRNHTKRIQDPTEDAEAEDTPRKRLRTDKHSLRKRDTITDKTARTSSYKAMEAASSLKSQAATKNLSDACKPLKKRNRASTAASSALGFSKSSSPSASLTENEVSDSPGDEPSESPYESADETQTEVSVSSKKSERGVTAKKEYVCQLCEKPGSLLLCEGPCCGAFHLACLGLSRRPEGRFTCSECASGIHSCFVCKESKTDVKRCVVTQCGKFYHEACVKKYPLTVFESRGFRCPLHSCVSCHASNPSNPRPSKGKMMRCVRCPVAYHSGDACLAAGCSVIASNSIICTAHFTARKGKRHHAHVNVSWCFVCSKGGSLLCCESCPAAFHPDCLNIEMPDGSWFCNDCRAGKKLHFQDIIWVKLGNYRWWPAEVCHPKNVPPNIQKMKHEIGEFPVFFFGSKDYYWTHQARVFPYMEGDRGSRYQGVRGIGRVFKNALQEAEARFREIKLQREARETQESERKPPPYKHIKVNKPYGKVQIYTADISEIPKCNCKPTDENPCGFDSECLNRMLMFECHPQVCPAGEFCQNQCFTKRQYPETKIIKTDGKGWGLVAKRDIRKGEFVNEYVGELIDKEECMARIKHAHENDITHFYMLTIDKDRIIDAGPKGNYSRFMNHSCQPNCETLKWTVNGDTRVGLFAVCDIPAGTELTFNYNLDCLGNEKTVCRCGASNCSGFLGDRPKTSTTLSSEEKGKKTKKKTRRRRAKGEGKRQSEDECFRCGDGGQLVLCDRKFCTKAYHLSCLGLGKRPFGKWECPWHHCDVCGKPSTSFCHLCPNSFCKEHQDGTAFSCTPDGRSYCCEHDLGAASVRSTKTEKPPPEPGKPKGKRRRRRGWRRVTEGK

SEQ ID NO: 4 - protein, 704 aa, Homo sapiens
MAAKKEYVCQLCEKTGSLLLCEGPCCGAFHLACLGLSRRPEGRFTCTECASGIHSCFVCKESKMEVKRCVVNQCGKFYHEACVKKYPLTVFESRGFRCPLHSCMSCHASNPSNPRPSKGKMMRCVRCPVAYHGGDACLAAGCSVIASNSIICTGHFTARKGKRHHTHVNVSWCFVCSKGGSLLCCEACPAAFHPDCLNIEMPDGSWFCNDCRAGKKLHFQDIIWVKLGNYRWWPAEVCHPKNVPPNIQKMKHEIGEFPVFFFGSKDYYWTHQARVFPYMEGDRGSRYQGVRGIGRVFKNALQEAEARFNEVKLQREARETQESERKPPPYKHIKVNKPYGKVQIYTADISEIPKCNCTPTDENPCGSDSECLNRMLMFECHPQVCPAGEYCQNQCFTKRQYPETKIIKTDGKGWGLVAKRDIRKGEFVNEYVGELIDEEECMARIKYAHENDITHFYMLTIDKDRIIDAGPKGNYSRFMNHSCQPNCETLKWTVNGDTRVGLFAVCDIPAGTELTFNYNLDCLGNEKTVCRCGASNCSGFLGDRPKTSASLSSEEKGKKAKKKTRRRRAKGEGKRQSEDECFRCGDGGQLVLCDRKFCTKAYHLSCLGLGKRPFGKWECPWHHCDVCGKPSTSFCHLCPNSFCKEHQDGTAFRSTQDGQSYCCEHDLRADSSSSTKTEKPFPESLKSKGKRKKRRCWRRVTDGK

SEQ ID NO: 5 - protein, 647 aa, Homo sapiens
MEFSIKQSPLSVQSVVKCIKMKQAPEILGSANGKTPSCEVNRECSVFLSKAQLSSSLQEGVMQKFNGHDALPFIPADKLKDLTSRVFNGEPGAHDAKLRFESQEMKGIGTPPNTTPIKNGSPEIKLKITKTYMNGKPLFESSICGDSAADVSQSEENGQKPENKARRNRKRSIKYDSLLEQGLVEAALVSKISSPSDKKIPAKKESCPNTGRDKDHLLKYNVGDLVWSKVSGYPWWPCMVSADPLLHSYTKLKGQKKSARQYHVQFFGDAPERAWIFEKSLVAFEGEGQFEKLCQESAKQAPTKAEKIKLLKPISGKLRAQWEMGIVQAEEAASMSVEERKAKFTFLYVGDQLHLNPQVAKEAGIAAESLGEMAESSGVSEEAAENPKSVREECIPMKRRRRAKLCSSAETLESHPDIGKSTPQKTAEADPRRGVGSPPGRKKTTVSMPRSRKGDAASQFLVFCQKHRDEVVAEHPDASGEEIEELLRSQWSLLSEKQRARYNTKFALVAPVQAEEDSGNVNGKKRNHTKRIQDPTEDAEAEDTPRKRLRTDKHSLRKRDTITDKTARTSSYKAMEAASSLKSQAATKNLSDACKPLKKRNRASTAASSALGFSKSSSPSASLTENELLWEPTPVKLDLNPAALYCT

SEQ ID NO: 6 - DNA, 30 bp, Artificial
  Primer
gtctgagatcctttgaatttaatttatgga

SEQ ID NO: 7 - DNA, 30 bp, Artificial
  Primer
gcgctgccacagggcaaagtccagttctac

SEQ ID NO: 8 - DNA, 19 bp, Artificial
  shRNA
acatgctcactatagacaa

SEQ ID NO: 9 - DNA, 21 bp, Artificial
  shRNA
cggaaagccaagttcaccttt

SEQ ID NO: 10 - DNA, 19 bp, Artificial
  shRNA
gaataagccttacgggaaa

SEQ ID NO: 11 - DNA, 19 bp, Artificial
  shRNA
gctataactcacataatgt

SEQ ID NO: 12 - DNA, 8 bp, Artificial
  shRNA
aagctttt

SEQ ID NO: 13 - protein, 37 aa, Homo sapiens
DGKGWGLVAKRDIRKGEFVNEYVGELIDEEECMARIK

SEQ ID NO: 14 - protein, 37 aa, Homo sapiens
DVAGWGIFIKDPVQKNEFISEYCGEIISQDEADRRGK